(19) United States
(12) Patent Application Publication
(10) Pub. No.: US 2022/0047204 A1
(43) Pub. Date: Feb. 17, 2022

(54) Methods, Computer-Readable Media and Devices for Producing an Index

(71) Applicants: JÓHANNSSON Magnús, Oslo (NO); Mentis Cura, Oslo (NO)

(72) Inventors: Magnús Jóhannsson, Oslo (NO); Kristinn Johnsen, Oslo (NO); Ivar Meyvantsson, Oslo (NO)

(21) Appl. No.: 17/277,234

(22) PCT Filed: Sep. 23, 2019
(86) PCT No.: PCT/US2019/052468

§ 371 Date: Mar. 17, 2021

Related U.S. Application Data
(60) Provisional application No. 62/735,681, filed on Sep. 24, 2018.

(51) Int. Cl. A61B 5/384; A61B 5/00; G16H 50/30; G16H 20/70; G16H 50/20; G16H 15/00

(52) U.S. Cl. CPC A61B 5/384; A61B 5/742; A61B 5/4088; G16H 15/00; G16H 50/30; G16H 20/70; G16H 50/20; A61B 5/4836

(57) ABSTRACT

Provided are computer-implemented methods for producing an index. The methods include conditioning electroencephalographic (EEG) signals present in an EEG recording previously obtained from an individual, e.g., an individual having dementia. In certain embodiments, the methods further include determining frequency domain features from the conditioned EEG signals, and determining connectivity features from the frequency domain features, where the connectivity features include connectivity features determined from a frequency range of from 35 Hz to 45 Hz divided into two or more sub-bands. The methods further include producing an index calculated at least in part as a function of one or more of the connectivity features determined from a frequency range of from 35 Hz to 45 Hz divided into sub-bands with varying contribution to the calculation of the index. Also provided are computer readable media and computer devices that find use, e.g., in practicing the methods of the present disclosure.

### CROSS-REFERENCE TO RELATED APPLICATIONS

This application claims the benefit of U.S. Provisional Patent Application No. 62/735,681, filed Sep. 24, 2018, which application is incorporated herein by reference in its entirety.

### INTRODUCTION

Electroencephalography (EEG) is an electrophysiological technique for the recording of electrical activity arising from the brain. Given its exquisite temporal sensitivity, EEG is useful in the evaluation of dynamic cerebral functioning. EEG finds use in the evaluation of clinical indications such as epilepsy, monitoring anesthesia during surgical procedures, and the like.

Clinical EEG assessment is mainly performed through visual inspection, identifying clear signs of pathology, while usually not considering other quantifiable measures. Potentially relevant features that are not immediately visible, such as power modulations, connectivity changes, or sparse small amplitude phenomena, may thus be overlooked. Quantitative EEG (qEEG) is a method of analyzing the electrical activity of the brain to derive quantitative patterns that may correspond to diagnostic information and/or cognitive deficits. qEEG analysis may therefore be helpful in the clinical context. For example, it is known that decreases of alpha and beta power and increases of the delta and theta frequencies are related to brain pathology and general cognitive decline. See, e.g., Dierks et al. (1995) Neural Transm Gen Sect 99:55-62; Kwak Y T (2006) J Clin Neurophysiol 23(5):456-61; and Anghinah et al. (2011) Arq Neuropsiquiatr 69(6) :871-4.

Resting EEG activity can predict future cognitive decline or conversion to dementia in mild cognitive impairment (MCI) subjects with high accuracy. See, e.g., Jelic et al. (2000) Neurobiol Aging 21(4):533-40; Prichep L S (2007) Ann N Y Acad Sci 1097:156-67; and Rossini et al. (2006) Neuroscience 143(3):793-803. In addition, studies suggest that spectral analysis can be used to distinguish Alzheimer's Disease (AD) from other dementias. See, e.g., Klassen et al. (2011) Neurology 77(2):118-24; Gawel et al. (2009) J Neurol Sci 283(1-2):127-33; and Schreiter Gasser et al. (2008) Clin Neurophysiol 119(10):2255-9. These studies use various EEG markers such as spectral power, coherence, and frequency of rhythms in delta, theta, alpha, or beta bands, which are considered valuable markers for group classification according to several studies. See, e.g., Kwak Y T (2006) J Clin Neurophysiol 23(5):456-61; and Fraga et al. (2013) PLoS One 8(8):e72240. Many EEG studies using qEEG analysis for classification (e.g., of AD) differ on the test-paradigm, sample size, methods, features extracted, and classification models. See, e.g., Dauwels et al. (2010) Curr Alzheimer Res 7(6):487-505.

### SUMMARY

Provided are computer-implemented methods for producing an index. The methods include conditioning electroencephalographic (EEG) signals present in an EEG recording previously obtained from an individual, e.g., an individual having dementia. In certain embodiments, the methods further include determining frequency domain features from the conditioned EEG signals, and determining connectivity features from the frequency domain features, where the connectivity features include connectivity features determined from a frequency range of from 35 Hz to 45 Hz divided into two or more sub-bands. The methods further include producing an index calculated at least in part as a function of one or more of the connectivity features determined from a frequency range of from 35 Hz to 45 Hz divided into two or more sub-bands with varying contribution to the calculation of the index. Also provided are computer readable media and computer devices that find use, e.g., in practicing the methods of the present disclosure.

### BRIEF DESCRIPTION OF THE FIGURES

FIG. 1: A flow diagram of a method according to some embodiments of the present disclosure.

FIG. 2: A flow diagram of a method according to some embodiments of the present disclosure.

FIG. 3: Graphs showing principal components and channel pairs employed to produce an index according to an embodiment of the present disclosure.

FIG. 4: An example of a type of report that may be generated according to some embodiments of the present disclosure.

FIG. 5: Scatter plots contrasting the training performance (x-axis) with the validation performance (y-axis) for two different sets allowing 0.5-35 Hz (LP 35 Hz—left panel) and 0.5-45 Hz (LP 45 Hz—right panel).

FIG. 6: Histograms showing the distribution of AUC values for two sets of classifiers (LP-35Hz and LP-45Hz) applied to the training set (left panel) and an independent validation set (right panel).

FIG. 7: Histograms showing the distribution of AUC values for two sets of classifiers (LP-35 Hz and LP-45 Hz) applied to the training set (left panel) and an independent validation set (right panel), where only those classifiers with AUC >0.92 when applied to the training set are considered.

FIG. 8: A flow diagram of a method according to some embodiments of the present disclosure.

FIG. 9: A flow diagram of a method according to some embodiments of the present disclosure.

FIG. 10: Comparison of the statistics of the classifier candidate's performance in terms of the estimated AUC revealing a significant performance benefit of including the sex of the individual as a feature.

FIG. 11: ROC curves showing significantly greater sensitivity and specificity when the sex of the individual is included as a feature.

FIG. 12: AUC statistics for genetic evolution-generated classifiers revealing a significant benefit of including the age of the individual as a feature.

FIG. 13: Response curves illustrating how equipment from different manufacturers and type respond to signals at different frequencies within the relevant frequency range for EEG recordings.

### DETAILED DESCRIPTION

Provided are computer-implemented methods for producing an index. The methods include conditioning electroencephalographic (EEG) signals present in an EEG recording previously obtained from an individual, e.g., an individual having dementia. According to some embodiments, the methods further include determining frequency domain features from the conditioned EEG signals, and determining connectivity features from the frequency domain features, where the connectivity features include connectivity features determined from a frequency range of from 35 Hz to 45 Hz divided into two or more sub-bands. The methods further include producing an index calculated at least in part as a function of one or more of the connectivity features determined from a frequency range of from 35 Hz to 45 Hz divided into two or more sub-bands with varying contribution to the calculation of the index. Also provided are computer readable media and computer devices that find use, e.g., in practicing the methods of the present disclosure.

Before the methods, computer-readable media and devices of the present disclosure are described in greater detail, it is to be understood that the methods, computer-readable media and devices are not limited to particular embodiments described, as such may, of course, vary. It is also to be understood that the terminology used herein is for the purpose of describing particular embodiments only, and is not intended to be limiting, since the scope of the methods, computer-readable media and devices will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed. The upper and lower limits of these smaller ranges may independently be included in the smaller ranges and are also encompassed, subject to any specifically excluded limit in the stated range. Where the stated range includes one or both of the limits, ranges excluding either or both of those included limits are also included.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods, computer-readable media and devices belong. Although any methods, computer-readable media and devices similar or equivalent to those described herein can also be used in the practice or testing of the methods, computer-readable media and devices, representative illustrative methods, computer-readable media and devices are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods, computer-readable media and devices are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods, computer-readable media and devices, which are, for clarity, described in the context of separate embodiments, may also be provided in combination in a single embodiment. Conversely, various features of the methods, computer-readable media and devices, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present methods, computer-readable media and devices and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods, computer-readable media and devices. Any recited method can be carried out in the order of events recited or in any other order that is logically possible.

### Methods

As summarized above, provided are methods of producing an index. The present methods are computer-implemented—that is, one or more steps are performed by one or more processors of one or more computer devices. The index produced according to the methods finds use in a variety of contexts. In certain aspects, the index is a dementia index which finds use, e.g., in diagnosing an individual as having a particular type of dementia, staging the individual's dementia, monitoring the progression of the individual's dementia, predicting the onset of dementia in an individual, and combinations thereof. For example, as demonstrated in the Experimental section below, the index produced according to the subject methods may be used to differentially diagnose an individual as having a Lewy Body Dementia (LBD—e.g., an individual having Dementia with Lewy Bodies (DLB) or Parkinson's Disease Dementia (PDD)) versus Alzheimer's Disease (AD) dementia. Previous approaches have primarily focused on alpha and delta bands with various measures, and some on measures of theta band activity. The methods of the present disclosure are based in part on the discovery of the importance of including higher frequencies than previously contemplated in order to achieve robust differentiation of individuals having DLB/PDD from individuals having AD in routine clinical practice.

Conditioning of EEG Signals

The present methods include conditioning, using one or more processors, electroencephalographic (EEG) signals present in an EEG recording previously obtained from an individual, e.g., an individual having dementia. When the individual has dementia, the individual may have already been diagnosed as having dementia when the EEG recording was obtained. In some embodiments, when the EEG recording was obtained, the individual had not been diagnosed as having dementia. By “conditioning” is meant the removal of certain artifacts and/or noise from the EEG signal. The artifacts and/or noise may arise from non-cerebral electrical activity, including but not limited to, eye blinks, eye flutter, lateral eye movement, slow/rowing eye movements, lateral rectus spikes, muscle (EMG or myogenic) movement, swallowing, chewing, or from the outside environment such as powerline interference or any combination thereof. In some embodiments, conditioning the EEG signals includes signal filtering, summing, squaring, subtracting, amplifying, or any combination thereof. The conditioning may include applying a filter to the EEG signals. Examples of filters which may be applied include a lattice filter, a FIR (finite impulse response) filter and an IIR (infinite impulse response) filter. Non-limiting examples of IIR filters which may be applied to the EEG signals include a Butterworth IIR filter, a Chebyshev IIR filter, and an elliptic IIR filter. In some embodiments, the EEG signals are conditioned using a high-pass filter, a low-pass filter, or both. For example, the EEG signals may be conditioned using a high-pass and subsequently a low-pass filter, or vice versa.

Feature Extraction

The subject methods further include extracting features from the conditioned EEG signals. In some embodiments, the EEG signals are analyzed in segments. In certain aspects, the duration of the segments is from 0.5 to 5 seconds, such as from 0.5 to 4 seconds or from 1 to 3 seconds, e.g., about 2 seconds. When the EEG signals are analyzed in segments, the segments may be non-overlapping or overlapping. In certain aspects, the methods include analyzing overlapping segments, where the segments overlap by from 0.25 to 2 seconds, such as from 0.5 to 1.5 seconds, e.g., about 1 second. By way of example, the EEG signals may be analyzed in about 2 second segments overlapping by about 1 second.

Feature extraction may begin using an approach such as time frequency distributions (TFD), Fast Fourier Transform (FFT), eigenvector methods (EM), wavelet transform (WT), auto regressive method (ARM), and the like. In certain aspects, the present methods include determining, using the one or more processors, frequency domain features from the conditioned EEG signals. An example approach for determining frequency domain features is by Fast Fourier Transform (FFT) (see, e.g., Oppenheim and Schaffer (1999) Discrete-time signal processing. Prentice Hall, London) to transform the signals from the time domain into the frequency domain.

The methods of the present disclosure further include determining, using the one or more processors, connectivity features from the frequency domain features. Both connectivity and coherence are functionals of the covariance. To capture the degrees of freedom of quantities derived as functionals of covariance, the frequency dependent covariances and cross channel covariances of the signals are considered. This is what is meant by “connectivity features”. Determining connectivity features may include calculating EEG connectivity (or “coherence”) features, which indicate the degree of similarity of the EEG recorded at two sensors. Coherence ranges from 0 to 1. If the phase—rising and falling—of the two signals tend to be similar over time, then it suggests functional connectivity—that is, the two areas of the brain are working together. In certain aspects, the connectivity features are determined for each frequency band separately. In some embodiments, determining frequency domain features and/or determining connectivity features is achieved by principal component analysis (PCA).

Index Calculation

The index may be calculated as a function of any suitable number of connectivity features and, optionally, frequency domain features. In some embodiments, the index is calculated as a function of from 5 to 50 total features, such as from 5 to 40, from 5 to 35, from 5 to 30, or from 5 to 25 (e.g., from 5 to 20) total features, e.g., 5, 6, 7, 8, 9, 10, 11, 12, 13, 14, 15, 16, 17, 18, 19, 20, 21, 22, 23, 24 or 25 total features. In certain aspects, the index is calculated as a function of 50 or fewer, 40 or fewer, 30 or fewer, 25 or fewer, 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, or 5 or fewer total features.

According to some embodiments of the methods of the present disclosure, the index is calculated at least in part as a function of one or more (e.g., 2 or more, 3 or more, 4 or more, 5 or more, etc.) of the connectivity features determined from a frequency range of from 35 Hz to 45 Hz divided into two or more sub-bands with varying contribution to the calculation of the index. For example, when the index is calculated at least in part as a function of two connectivity features determined from a frequency range of from 35 Hz to 45 Hz divided into two or more sub-bands, the first and second connectivity features have a varied contribution to the index, e.g., the first connectivity feature may be given a greater weight for purposes of calculating the index compared to the second connectivity feature. The same principle may apply when the index is calculated at least in part as a function of 3 or more, 4 or more, 5 or more, 6 or more, 7 or more, 8 or more, 9 or more, or 10 or more connectivity features, where at least 2 of the connectivity features have a varying contribution to the calculation of the index.

In certain aspects, the index is based on a linear combination of the connectivity features. By “linear combination” is meant an equation is used in which each connectivity feature is multiplied by a constant and the products are summed. Examples of such linear combinations and constants are provided below. The index may also be based on a higher order function, such as a polynomial or exponential.

As summarized above, one or more of the connectivity features from which the index is calculated may be from a frequency range of from 35 Hz to 45 Hz divided into two or more sub-bands with varying contribution to the calculation of the index. As noted above, previous approaches have primarily focused on alpha and delta bands with various measures, and some on measures of theta band activity. The methods of the present disclosure are based in part on the discovery of the importance of including higher frequencies (e.g., from 35 Hz to 45 Hz) than previously contemplated in order to achieve, e.g., robust differentiation of individuals having DLB/PDD from individuals having AD in routine clinical practice.

The frequency resolution with which the width of the sub-bands are defined may vary. In some embodiments, the two or more sub-bands in a frequency range of from 35 Hz to 45 Hz are defined with a frequency resolution of from 0.1 to 10 Hz, such as from 0.2 to 5 Hz, from 0.3 to 2 Hz, or from 0.4 to 2 Hz, e.g., 0.5 to 1 Hz. In certain embodiments, the sub-bands are defined with a frequency resolution of from 0.5 Hz.

In certain embodiments, in addition to one or more of the connectivity features, the index is calculated as a function of one or more of the frequency domain features. According to some embodiments, the one or more frequency domain features are from a frequency range of from 35 Hz to 45 Hz divided into two or more sub-bands with varying contribution to the calculation of the index.

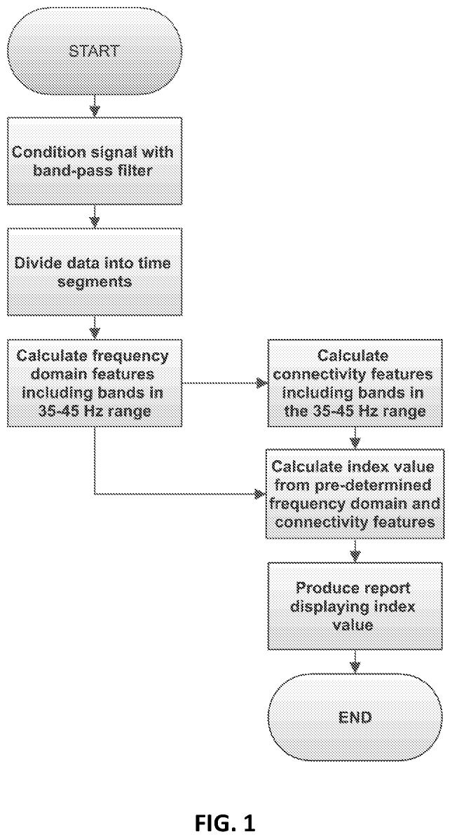

A flow chart illustrating a method according to some embodiments of the present disclosure is provided in FIG. 1. In this example, the EEG signal is conditioned with a band-pass filter to eliminate baseline shift and high-frequency noise. The conditioned data are divided into time segments. Frequency domain features are determined from the time segmented data and, additionally, inter-channel and intra-channel connectivity features are calculated based on the frequency domain features. The magnitude of the frequency domain features and connectivity results for each frequency band form the collection of features characterizing the individual recording. Statistical methods are used to estimate the value of each feature over the series of time segments. An index is calculated from a pre-defined subset of features using a pre-defined formula with pre-defined coefficients, examples of which are described below. A report may be produced to present the index value result together with relevant information.

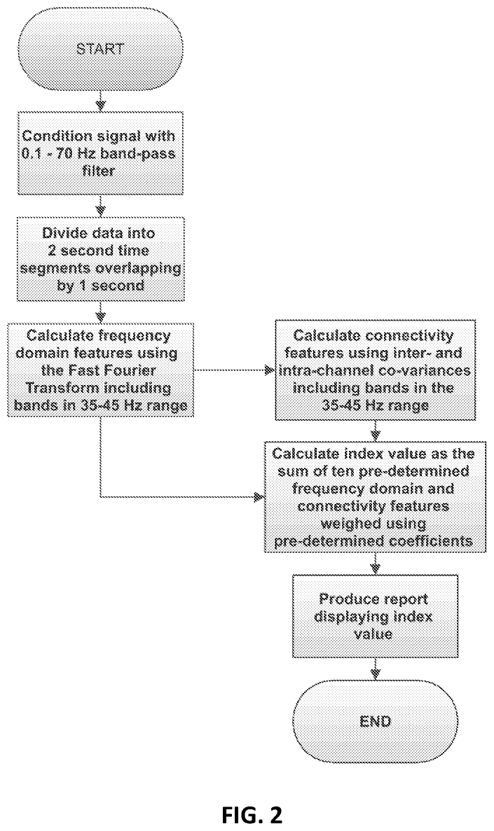

Shown in FIG. 2 is a flow chart which provides non-limiting examples of how the steps shown in the flowchart of FIG. 1 may be performed. In this example, the EEG signals are conditioned using a 0.1-70 Hz band-pass filter and the conditioned signals are divided into 2 second time segments overlapping by 1 second, the frequency domain features including bands in the 35-45 Hz range are calculated using the Fast Fourier Transform (FFT), and the connectivity features including bands in the 35-45 Hz range are calculated using inter- and intra-channel covariances. Also in this example, the index value is calculated as the sum of ten pre-determined frequency domain and connectivity features weighed using pre-determined coefficients.

Index Calculation Using Sex and/or Age as a Feature

Aspects of the present disclosure further include computer-implemented methods for producing an index, where the sex of the individual is used as a feature, the age of the individual is used as a feature, or the sex and age of the individual are used as features, in the index calculation.

According to some embodiments, provided are methods that comprise conditioning the EEG signals, determining frequency domain features from the conditioned EEG signals, determining connectivity features from the frequency domain features, and calculating one or more connectivity features (each of which may be as described elsewhere herein), and where the index is further calculated as a function of the age of the individual, the sex of the individual, or both.

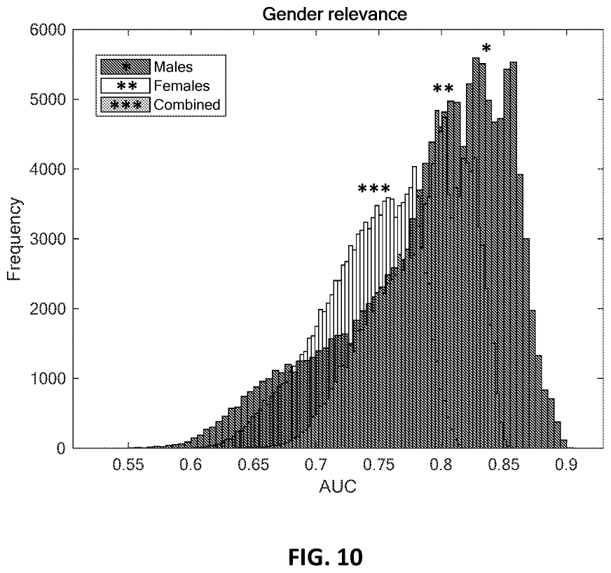

As demonstrated in the Experimental section and illustrated in the Drawings herein, the inventors have determined that including the sex and/or age of the individual results in a performance benefit to the index. See, e.g., Example 2 and FIGS. 10-12.

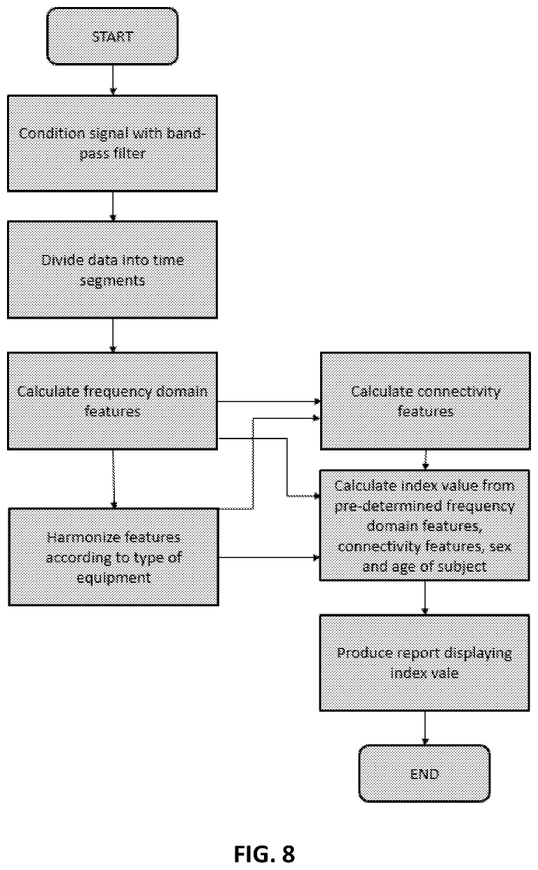

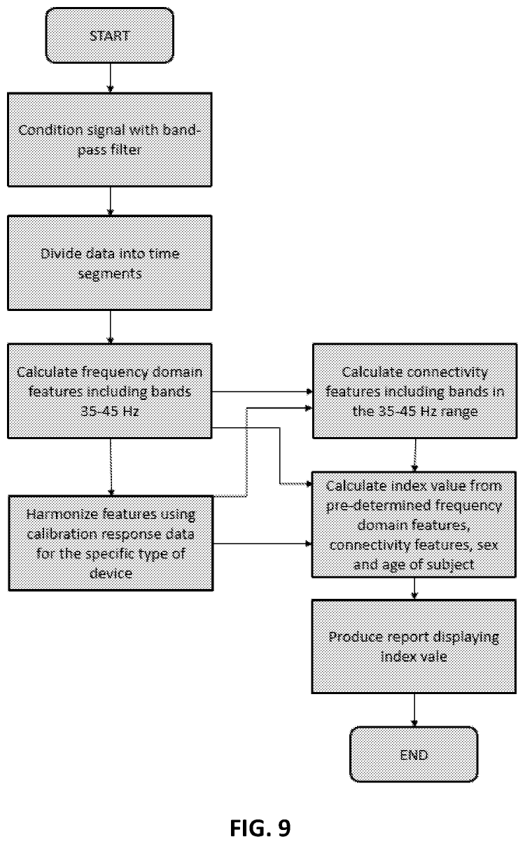

Flow diagrams showing an example method (and variations thereof) of the present disclosure that take the age and sex of the individual into account when producing the index are provided in FIGS. 8 and 9. In this example shown in FIG. 8, the EEG signal is conditioned with a band-pass filter to eliminate baseline shift and high-frequency noise. The conditioned data are divided into time segments. Frequency domain features are determined from the time segmented data and, additionally, inter-channel and intra-channel connectivity features are calculated based on the frequency domain features. The magnitude of the frequency domain features and connectivity results for each frequency band form the collection of features characterizing the individual recording. Statistical methods are used to estimate the value of each feature over the series of time segments. An index is calculated from a pre-defined subset of features—including the sex and age of the individual. Also in this example, frequency domain features are harmonized based on the type of EEG recording equipment used to obtain the EEG recording (details of which are described elsewhere herein). A report may be produced to present the index value result together with relevant information.

Shown in FIG. 9 is a flow chart which provides non-limiting examples of how the steps shown in the flowchart of FIG. 8 may be performed. In this example, the frequency domain features include bands in the 35-45 Hz range, and connectivity features including bands in the 35-45 Hz range are calculated. Also in this example, the frequency domain features are harmonized using calibration response data for the specific type of EEG recording equipment used to obtain the EEG recording.

Harmonization of Frequency Domain Features

Aspects of the present disclosure further include computer-implemented methods for producing an index, where the frequency domain features are harmonized based on the type of EEG recording equipment used to obtain the EEG recording. The “type” of the EEG recording equipment may refer to the manufacturer of the equipment, the model of the EEG equipment, or both. By “harmonizing” the frequency domain features is meant rescaling the frequency domain features derived from EEG recordings from different EEG amplifiers according to the measured frequency dependent power response from that particular EEG amplifier, thereby making the estimated quantitative values of the frequency domain features independent of the EEG amplifier used to obtain the EEG recording.

According to some embodiments, provided are methods that comprise conditioning the EEG signals and determining frequency domain features from the conditioned EEG signals (each of which may be as described elsewhere herein), and where method further includes harmonizing the frequency domain features based on the type of EEG recording equipment used to obtain the EEG recording. Such methods further include producing an index calculated at least in part as a function of the harmonized frequency domain features, and optionally one or more connectivity features calculated from one or more of the harmonized frequency domain features. In certain embodiments, the frequency domain features are harmonized using calibration response data for the specific type of EEG recording equipment used to obtain the EEG recording.

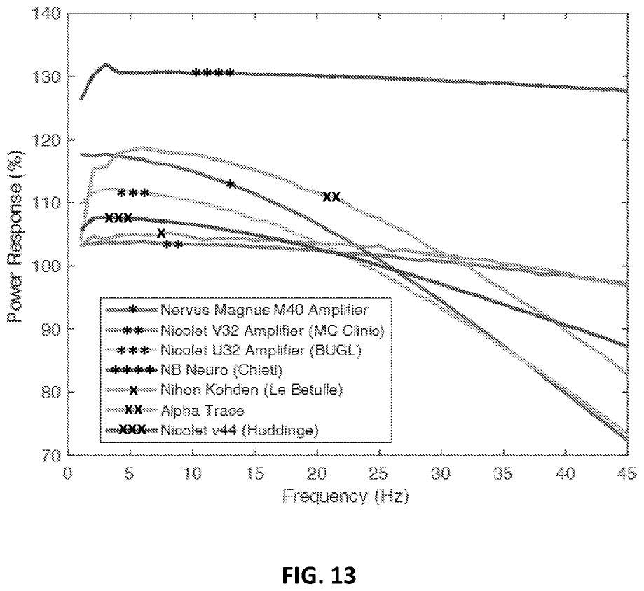

Details regarding a non-limiting approach for harmonizing the frequency domain features are provided in Example 3 below. FIG. 13 illustrates how equipment from different manufacturers and type respond to signals at different frequencies within the relevant frequency range for EEG recordings. The characteristic response curves are measured by feeding a sinusoidal signal of known amplitude and frequency into the equipment with a signal generator. This is done by fixing amplitude of the signal and then stepping through the relevant frequency range at, say steps of 0.5 Hz. The amplitude measured by the equipment is then compared to the reference signal and the power response is deduced by the squared ratio of the measured signal to the reference signal at that frequency. For a specific piece of equipment, this procedure results in power response curve pᵢ where i is the frequency. Examples of response curves are shown in FIG. 13. In this example, harmonization of features estimated by the equipment is achieved by scaling the resulting Fourier components stored in the SPC format, σ_{cij}. The scaling is achieved by σ_{cij}^{sc}=σ_{cij}/\sqrt{pᵢ}. Further estimates based on the FFT components are then done using the scaled components.

Flow diagrams illustrating non-limiting examples of how harmonization of frequency domain features may be incorporated into methods of producing an index are provided in FIGS. 8 and 9.

Exemplary Non-limiting Method

The following is a non-limiting example of how an index may be produced according to the methods of the present disclosure.

EEG signals present in an EEG recording previously obtained from an individual having dementia are conditioned by filtering using a Butterworth filter. The EEG recording was previously produced using a 19 electrode setup, where the electrodes are ordered as: Fp1, Fp2, F3, F4, C3, C4, P3, P4, O1, O2, F7, F8, T3, T4, T5, T6, Fz, Cz, and Pz. The EEG is analyzed in segments, where the segments are 2 second segments overlapping by 1 second. Subsequent to conditioning, a frequency-domain transform is determined by Fast Fourier Transform (FFT). The FFT spectrum is then evaluated for each segment. This evaluation is done in the average montage, where the amplitude is evaluated relative to the grand average over all electrode values. Spectral resolution is then 0.5 Hz. The frequency domain transformation for all segments in a recording are then stored in spectral power coherence (SPC) format, one line for each segment. 90 frequency points are stored for 0.5 Hz to 45 Hz in 0.5 Hz intervals. The complex values of the corresponding Fourier components are stored as alternating real and imaginary parts of the coefficients, resulting in a data matrix. This is the core (N×90) data matrix format assumed as base input for subsequent analysis for each electrode. For a recording with M electrodes, the core data matrices are concatenated left to right, resulting in a grand (N×90M) matrix containing all the data for the recording.

Continuing with this example, let the Fourier components stored in the SPC format be denoted by σ_{cij}, where c∈{1, . . . , 19} indicates the channel, i∈{1, . . . N} denotes the segment, and J∈{1, . . . 90} refers to the frequency. The features relied upon are on one hand qEEG features related to each channel. Then the full spectrum for each channel C becomes

ρ_{c}^{ij}=σ_{cij}*σ_{cij}^{†}

for segment i and frequency j. The full spectral resolution covariance between channels C and k features become

x_{ck}^{ij}=σ_{cij}*σₖᵢⱼ^{†}

for segment i and frequency i.

Continuing with this example, for the qEEG spectral features, bands are identified through optimization for each electrode or electrode pair, C, and band label, α, to define the final features used. This is done by reducing the degrees of freedom by applying PCA. First, PCA bands are selected according to the variance of the data which is explained. A finite number of PCAs are used for each C labeled by α. The PCAs used are referred to by, T_{cαi}, where i is the frequency. Then, the actual features considered for the index are

C_{cα}=Eᵢ{Σⱼ₌₁⁹⁰x_{c}^{ij}T_{cαj}},

where Eₖ{αₖ} is a robust estimate of αₖ. Here, the median is used.

Continuing with this example, the index is evaluated according to the Formula I=Cβ+ρ=χPβ+ρ, where P is a set of principal components corresponding to a specific channel pair or individual channels considered for classification, and where χ is all the possible inter-channel covariances or individual channel spectral powers. The matrix χ has a dimension of N×M where N is the number of channel pairs and M is the number of spectral points, taken into account, from the recording. The index is defined by the vector β, which may have only a finite number of non-zero elements, while ρ is a constant determining the decision-point for the classifier. For example, making certain that the optimal decision-point is a value of zero.

In certain aspects, one or more of the connectivity features utilize 1 or more, 2 or more, 3 or more, 4 or more, 5 or more, 6 or more, 7 or more, 8 or more, or each of the following channel pairs: F3-O2, F3-T5, F4-O2, C3-O1, P3-O2, P4-T5, T4-T6, T4-Fz, Pz-Pz, in any combination.

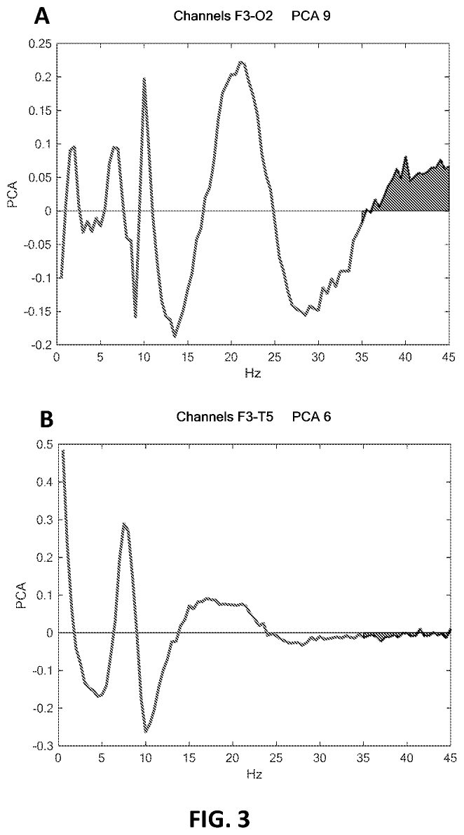

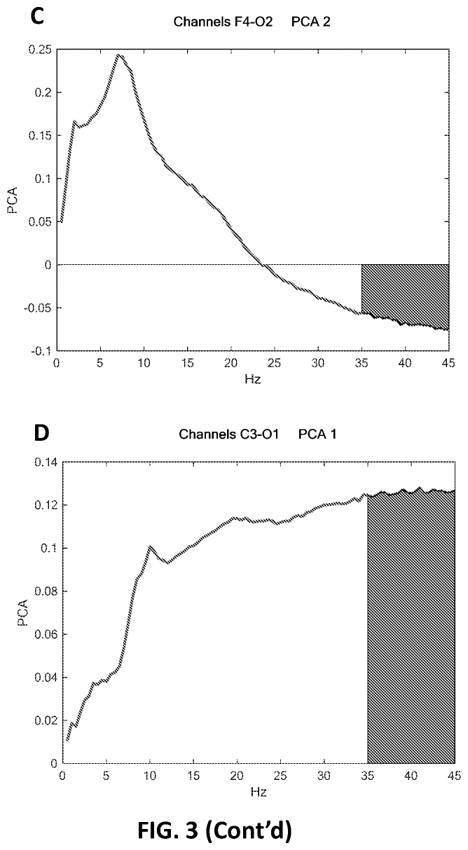

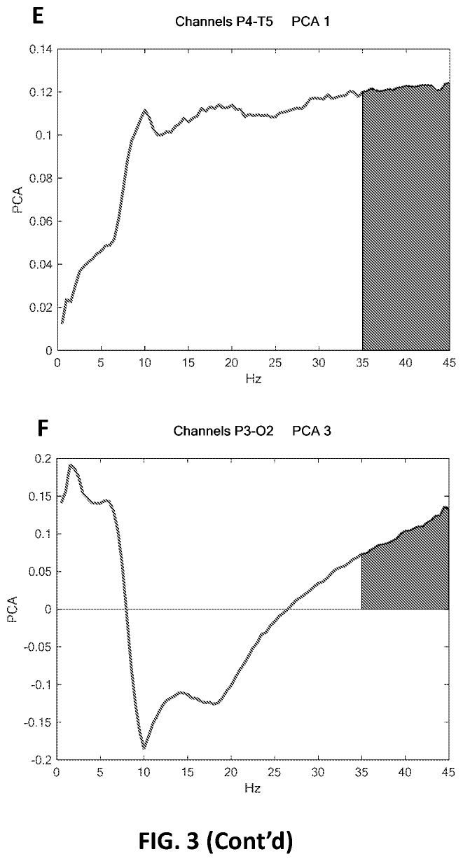

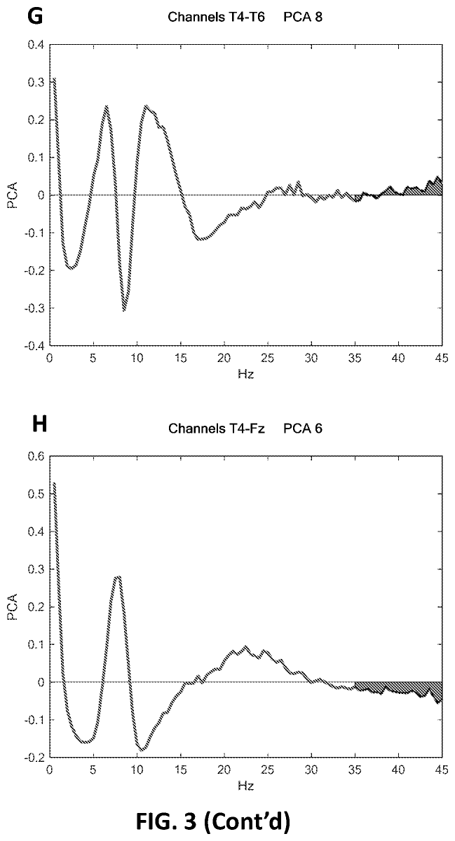

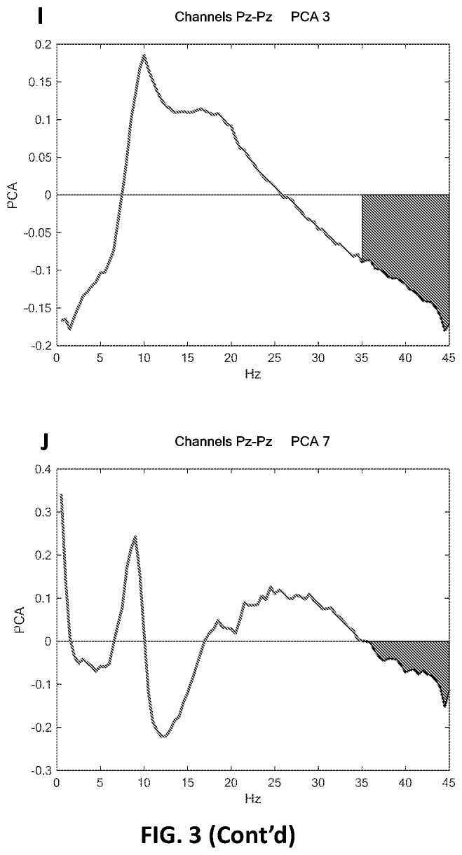

The following is a specific non-limiting example of use of a classifier that performs well in classification of Alzheimer's subjects versus subjects suffering from Dementia with Lewy Bodies, classifying a subject from each group. The classifier is chosen to rely on 10 channel pairs and associated principal components. The pairs are chosen by applying optimization on overall performance of the classifier. The index is calibrated such that a positive outcome indicates Alzheimer's, while a negative outcome indicates Dementia with Lewy Bodies. Table 1 lists the channel pairs used in the example and which principal component of the covariance the used features rely on. Also listed are the specific feature values for an Alzheimer's subject, the non-zero values of β entering the classifier, as well as the calibration constant ρ. The final column lists the contribution from each feature for this feature which are then all added up resulting in an index value of 2.40 consistent with an Alzheimer's subject. Table 2 lists the same for a specific subject having Dementia with Lewy Bodies, resulting in an index value of −0.40, consistent with Dementia with Lewy Bodies according to the classifier. All of the principal components for each channel pair applied for this classifier are illustrated in FIG. 3. The relevant bands in the frequency range 35-45 Hz are shaded, indicating their contribution to the index. Their contribution is significant in panels A, C, D, E, F, I and J of FIG. 3.

TABLE 1
Alzheimer's Disease

| Alzheimer's Subject | | Feature | | | Index |
|---|---|---|---|---|---|
| Channels | PCA | Value | Beta | rho | Contribution |
| F3-O2 | 9 | −0.13 | −0.76 | 0.44 | 0.54 |
| F3-T5 | 6 | 3.33 | 0.28 | 0.44 | 1.37 |
| F4-O2 | 2 | 27.69 | −0.25 | 0.44 | −6.35 |
| C3-O1 | 1 | 54.30 | 0.07 | 0.44 | 4.30 |
| P3-O2 | 3 | 6.73 | −0.24 | 0.44 | −1.16 |
| P4-T5 | 1 | 45.51 | 0.04 | 0.44 | 2.30 |
| T4-T6 | 8 | 1.53 | −0.37 | 0.44 | −0.12 |
| T4-Fz | 6 | 3.94 | 0.32 | 0.44 | 1.69 |
| Pz-Pz | 3 | −5.69 | 0.03 | 0.44 | 0.30 |
| Pz-Pz | 7 | 2.14 | −0.41 | 0.44 | −0.45 |
| | | | | Index | 2.40 |

TABLE 2
Dementia with Lewy Bodies

| Dementia with Lewy Bodies Subject | | | | | |
|---|---|---|---|---|---|
| | | Feature | | | Index |
| Channels | PCA | Value | Beta | rho | Contribution |
| F3-O2 | 9 | 0.21 | −0.76 | 0.44 | 0.28 |
| F3-T5 | 6 | 3.59 | 0.28 | 0.44 | 1.44 |
| F4-O2 | 2 | 27.81 | −0.25 | 0.44 | −6.38 |
| C3-O1 | 1 | 29.78 | 0.07 | 0.44 | 2.56 |
| P3-O2 | 3 | 8.23 | −0.24 | 0.44 | −1.52 |
| P4-T5 | 1 | 28.90 | 0.04 | 0.44 | 1.62 |
| T4-T6 | 8 | 0.89 | −0.37 | 0.44 | 0.12 |
| T4-Fz | 6 | 3.89 | 0.32 | 0.44 | 1.67 |
| Pz-Pz | 3 | −6.77 | 0.03 | 0.44 | 0.27 |
| Pz-Pz | 7 | 2.16 | −0.41 | 0.44 | −0.45 |
| | | | | Index | −0.40 |

Generating a Report

The methods of the present disclosure may further include generating a report that includes the index. In certain aspects, generating a report includes displaying the index on a display (e.g., a display of a desktop computer, laptop computer, television, tablet computer, smartphone, or the like) or printout. In some embodiments, when the index is displayed on a display, the index is displayed on the display of the computer device used to produce the index. Alternatively or additionally, the index may be displayed on the display of a computer device other than the computer device used to produce the index.

In certain aspects, when the methods include generating a report, the index may be displayed graphically in context with other information, non-limiting examples of which include historical results for the individual, illustrative theoretical distributions or distribution densities based on real-world data from a database of individuals having dementia and a database of individuals not having dementia, or any combination thereof. For example, the index may be displayed graphically in context with data from a database of individuals having a particular type of dementia and a database of individuals not having dementia. Alternatively or additionally, the index may be displayed graphically in context with data from a database of individuals having a first type of dementia and a database of individuals having a second type of dementia. Non-limiting examples of dementia which may make up the first and second types of dementia include a Lewy Body Dementia (including Dementia with Lewy Bodies and Parkinson's Disease Dementia), Dementia with Lewy Bodies, Parkinson's Disease Dementia, Alzheimer's Disease Dementia, Frontal Lobe Dementia, and Vascular Dementia. By way of example, the first type of dementia may be a Lewy Body Dementia and the second type of dementia may be Alzheimer's Disease Dementia.

In some embodiments, the methods further include displaying via a display a suggested diagnosis of dementia based at least in part on the index. The suggested diagnosis may suggest the individual has dementia versus not having dementia. In other aspects, the suggested diagnosis suggests the individual has a particular type of dementia versus not having dementia. In further aspects, the suggested diagnosis in the report suggests the individual has a first type of dementia (e.g., a Lewy Body Dementia) and not a second type of dementia, e.g., Alzheimer's Disease Dementia. That is, the suggested diagnosis in the report may be a suggested differential diagnosis. Examples of types of dementia for which the report may suggest a diagnosis include a Lewy Body Dementia (including Dementia with Lewy Bodies and Parkinson's Disease Dementia), Dementia with Lewy Bodies, Parkinson's Disease Dementia, Alzheimer's Disease Dementia, Frontal Lobe Dementia, and Vascular Dementia.

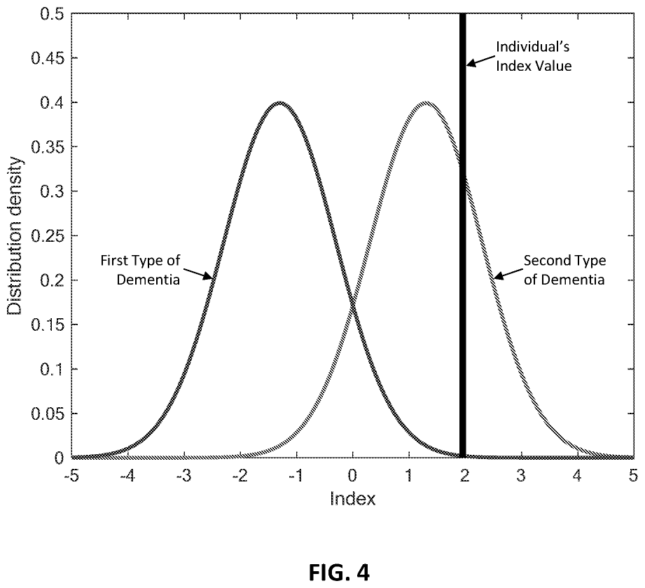

An example of a type of report that may be generated according to some embodiments is shown in FIG. 4. In this example, the index value of an individual is displayed graphically in context with data from a database of individuals having a first type of dementia and a database of individuals having a second type of dementia. This report includes a graph having index values ranging from −5 to 5 on its x-axis and distribution density on its y-axis. Shown is the distribution density across index values for individuals having a first type of dementia and the distribution density across index values for individuals having a second type of dementia. By way of example, the first type of dementia may be a Lewy Body Dementia and the second type of dementia may be Alzheimer's Disease Dementia. Non-limiting examples of dementia which may make up the first and second types of dementia include a Lewy Body Dementia (including Dementia with Lewy Bodies and Parkinson's Disease Dementia), Dementia with Lewy Bodies, Parkinson's Disease Dementia, Alzheimer's Disease Dementia, Frontal Lobe Dementia, and

Vascular Dementia. The individual's index value is indicated by a vertical line on the graph. Here, the individual's index value is 2, consistent with the individual having the second type of dementia. In some embodiments, based at least in part on the report, a diagnosis or suggested diagnosis of the individual having the second type of dementia may be made.

Index-Based Diagnosis

In certain aspects, the methods further include diagnosing the individual as having dementia based at least in part on the index. The diagnosing may include diagnosing the individual as having dementia generally (rather than a particular type of dementia) versus not having dementia. In some embodiments, the diagnosing includes diagnosing the individual as having a particular type of dementia versus not having dementia. In some embodiments, the diagnosing includes diagnosing the individual as having a first type of dementia and not a second type of dementia. That is, the diagnosing may include providing a differential dementia diagnosis. By way of example, based on the index, the methods may include providing a differential diagnosis where the individual is diagnosed as having a Lewy Body Dementia (generally, or Dementia with Lewy Bodies or Parkinson's Disease Dementia specifically) and not Alzheimer's Disease Dementia. As another example, the methods may include providing a differential diagnosis where the individual is diagnosed as having Alzheimer's Disease Dementia and not a Lewy Body Dementia (generally, or Dementia with Lewy Bodies or Parkinson's Disease Dementia specifically). Non-limiting examples of dementias which may be diagnosed according to the methods of the present disclosure include a Lewy Body Dementia,

Dementia with Lewy Bodies, Parkinson's Disease Dementia, Alzheimer's Disease Dementia, Frontal Lobe Dementia, and Vascular Dementia.

When the methods include diagnosing the individual, the diagnosis may be based on a report, such as any of the reports described above. For example, the diagnosis may be based a report in which the index is displayed on a display or printout, e.g., where the index is displayed graphically in context with data from a database of individuals having dementia and a database of individuals not having dementia, in context with data from a database of individuals having a particular type of dementia and a database of individuals not having dementia, in context with data from a database of individuals having a first type of dementia (e.g., a Lewy Body Dementia) and a database of individuals having a second type of dementia (e.g., Alzheimer's Disease Dementia), or the like.

When the methods include diagnosing the individual, the diagnosis may be based on—in addition to the index—a neuropsychiatric assessment of the individual, imaging of the individual's brain, analysis of a biomarker present in a body fluid of the individual, or any combination thereof.

In some embodiments, e.g., when the methods include diagnosing the individual, or providing a suggested diagnosis, the methods may include displaying, via a display, printout, or the like, a prompt to perform an assessment, e.g., a clinical assessment, to confirm or corroborate a diagnosis of dementia, a differential diagnosis of dementia, a suggested diagnosis of dementia, or a suggested differential diagnosis of dementia, e.g., one or more of a neuropsychiatric assessment of the individual, imaging of the individual's brain, and analysis of a biomarker present in a body fluid of the individual.

When the diagnosing includes neuropsychiatric assessment of the individual, any suitable assessment may be performed. In some embodiments, the neuropsychiatric assessment includes administering a cognitive test to the individual. For example, the neuropsychiatric assessment may include testing the individual using the Alzheimer's Disease Assessment Scale-Cognitive (ADAS-Cog) Subscale test. See, e.g., Skinner et al. (2012) Brain Imaging Behav. 6(4):10. When the diagnosing includes imaging of the individual's brain, non-limiting examples of imaging methodologies which may be employed include positron emission tomography (PET), single-photon emission computed tomography (SPECT), magnetic resonance imaging (MRI), or any combination thereof. The imaging may aid the diagnosis based on the index by revealing Lewy bodies (that is, abnormal deposits of alpha-synuclein) in the brain (aiding in the diagnosis of a Lewy Body Dementia), abnormal deposits of amyloid plaques and tau tangles in the brain (aiding in the diagnosis of Alzheimer's Disease Dementia), or the like. When the diagnosing includes analysis of a biomarker present in a body fluid of the individual, any suitable biomarker or panel of biomarkers may be analyzed. For example, the diagnosing may include analyzing the body fluid (e.g., blood, a particular blood component (e.g., plasma, serum, etc.), cerebrospinal fluid (CSF), and/or the like) for the presence of a marker diagnostic of a particular dementia, such as amyloid or amyloid-related proteins in the case of AD (see, e.g., Nakamura et al. (2018) Nature 554:249-254), alpha-synuclein protein in the case of LBD, etc. In certain aspects, analysis of a biomarker includes performing a genetic test in which the individual is tested for one or more genetic markers (e.g., mutations, single nucleotide polymorphisms (SNPs), and/or the like, associated (or causal) of a particular type of dementia.

Index-Based Treatment

When the methods include diagnosing the individual as having dementia generally or a type of dementia, the methods may further include recommending a dementia treatment for the individual based on the diagnosis. In certain aspects, when the methods include diagnosing the individual as having dementia generally or a type of dementia, the methods further include treating the individual's dementia based on the diagnosis. By “treat” or “treatment” is meant at least an amelioration of the symptoms associated with the dementia afflicting the individual, where amelioration is used in a broad sense to refer to at least a reduction in the magnitude of a parameter, e.g., symptom (e.g., memory loss, reduced motor control, and/or the like), associated with the dementia being treated. As such, treatment also includes situations where the dementia, or at least symptoms associated therewith, are completely inhibited, e.g., prevented from happening, or stopped, e.g. terminated, such that the individual no longer suffers from the dementia, or at least the symptoms that characterize the dementia.

The suggested and/or administered treatment may vary depending upon the diagnosis of the individual. Treatments may include one or more non-pharmaceutical treatments (e.g., cognitive exercises, etc.) and/or one or more pharmaceutical treatments. For example, a suitable pharmaceutical treatment includes administering a pharmaceutical (e.g., a biologic (e.g., antibody), small molecule, and/or the like) which is approved by the U.S. Food and Drug Administration (FDA), the European Medicines Agency (EMA), and/or the like, for treatment of dementia generally or a particular type of dementia with which the individual has been diagnosed based on the index. By way of example, when the individual is diagnosed as having Alzheimer's Disease Dementia based on the index, the treatment may include administering to the individual a cholinesterase inhibitor (e.g., galantamine, rivastigmine, donepezil, or the like), an N-methyl D-aspartate (NMDA) antagonist (e.g., memantine), Aricept®, the Exelon® patch, Namzaric®, a combination of Namenda® and Aricept®, and any combinations thereof. The manner in which the pharmaceutical is administered to the individual may vary depending upon the particular pharmaceutical. Depending on the particular pharmaceutical, suitable routes of administration include parenteral (e.g., intravenous, intracerebral, intracerebroventricular, intra-arterial, intraosseous, intramuscular, intrathecal, subcutaneous, etc.) administration, oral administration, etc.

Index-Based Staging/Assessment of Progression

The methods of the present disclosure may further include staging the individual's dementia based at least in part on the index. For example, based on the index, the individual's dementia may be assigned as stage 4 (moderate cognitive decline), stage 5 (moderately severe cognitive decline), stage 6 (severe cognitive decline (middle dementia)), or stage 7 (very severe cognitive decline (late dementia)), based on the Global Deterioration Scale for Assessment of Primary Degenerative Dementia (GDS). Alternatively, or additionally, based on the index, the individual's dementia may be assigned as CDR-0.5 (mild dementia—slight but consistent memory loss), CDR-1 (mild dementia—moderate memory loss), CDR-2 (moderate dementia), or CDR-3 (severe dementia), according to the Clinical Dementia Rating (CDR) scale. Alternatively, or additionally, based on the index, the individual's dementia may be assigned as stage 3 (early Alzheimer's Disease), stage 4 (mild Alzheimer's Disease), stage 5 (moderate Alzheimer's Disease), stage 6 (moderately severe Alzheimer's Disease), or stage 7 (severe Alzheimer's Disease), according to the Functional Assessment Staging Test (FAST) scale.

In some embodiments, the methods further include assessing the progression of the individual's dementia based at least in part on the index. For example, the index may be used to assign a stage to the individual's dementia, where the assigned stage is compared to an earlier or subsequent assigned stage of the individual's dementia. In certain aspects, the earlier or subsequent assigned stage may be based on an index produced according to the methods of the present disclosure. In some embodiments, the assigned stages are assigned based on one or more of the GDS, CDR, and FAST scales described above. Accordingly, in some embodiments, the subject methods are performed iteratively to monitor progression of the individual's dementia. When the methods are performed iteratively to monitor progression of the individual's dementia, producing the index may be performed on a daily basis, weekly basis, monthly basis (every 1, 2, 3, 4, 5, 6, 7, 8, 9, 10, or 11 months), or yearly basis, e.g., once per year, once per every 1.5 years, once per every 2 years, once per every 2.5 years, once per every 3 years, or the like.

Index-Based Prediction of Dementia Onset

Also provided are computer-implemented methods for producing an index, where the methods include conditioning, using one or more processors, electroencephalographic (EEG) signals present in an EEG recording previously obtained from an individual, determining, using the one or more processors, frequency domain features from the conditioned EEG signals, determining, using the one or more processors, connectivity features from the frequency domain features, where the connectivity features include connectivity features determined from a frequency range of from 35 Hz to 45 Hz divided into two or more sub-bands. Such methods further include producing, using the one or more processors, an index calculated at least in part as a function of one or more of the connectivity features determined from a frequency range of from 35 Hz to 45 Hz divided into two or more sub-bands with varying contribution to the calculation of the index. Such methods further include predicting the onset of dementia in the individual based at least in part on the index. By “predicting the onset of dementia” is meant the individual does not have dementia when the index is produced, but based on the index, the timing of onset of dementia in the individual is predicted. For example, based on the index, the onset of dementia in the individual may be predicted to be less than 1, 2 or fewer, 3 or fewer, 4 or fewer, 5 or fewer, 6 or fewer, 7 or fewer, 8 or fewer, 9 or fewer, or 10 or fewer years from the date that the index is produced.

Obtaining EEG Recordings

Any of the methods described herein may further include, prior to the conditioning, collecting EEG signals from the individual to obtain the EEG recording. In some embodiments, the EEG signals are collected using electrodes placed on the individual's scalp according to a standardized placement system, such as the “10-20” system or “International 10-20” system, or a modified placement system thereof. In the standard 10-20 system, 21 electrodes are located on the surface of the scalp. Reference points are nasion, which is the delve at the top of the nose, level with the eyes; and inion, which is the bony lump at the base of the skull on the midline at the back of the head. From these points, the skull perimeters are measured in the transverse and median planes. Electrode locations are determined by dividing these perimeters into 10% and 20% intervals. Three other electrodes are placed on each side equidistant from the neighboring points.

In some embodiments, the EEG signals are resting EEG signals which may be collected according to the following example. The individual is seated in a comfortable chair; electrodes are applied on the scalp using a standard pattern (IS-10-20 system) with 19 electrodes (one of the standard electrodes, located at the back of the head, is omitted relative to the standard to enable the individual to rest her/his head on the back of the chair); it is confirmed that the each electrode makes good contact by checking the impedance, which preferably is lower than 5 k-Ohm; the electrodes are connected to a biopotential amplifier suitable for clinical EEG measurements; the individual is instructed to close her/his eyes, relax, and not think about anything in particular; and recording an EEG registration of at least 1 minute (e.g., 2 or more minutes, 3 or more minutes, or the like) while the technician ensures that interruptions of the signal are avoided, such as neck muscle activity, eye movements, electrode lead movements, and/or external electrical noise. Signs of individuals falling asleep are specifically monitored and individuals gently awoken if needed. The total duration of the recording may be extended in order to ensure at least 3 minutes in total of recording segments unaffected by interruptions or artefacts. The collected EEG signals are captured on a suitable recording medium, such as a storage drive, e.g., a hard drive. The actual electrodes used could also be any subset of the 21 standard electrodes, as required to calculate the features of interest.

### Computer-Readable Media and Devices

Also provided are computer readable media for producing an index and devices including such computer readable media.

In certain aspects, provided is a non-transitory computer readable medium including instructions for producing an index, where the instructions, when executed by one or more processors, cause the one or more processors to condition electroencephalographic (EEG) signals present in an EEG recording previously obtained from an individual having dementia, determine frequency domain features from the conditioned EEG signals, determine connectivity features from the frequency domain features, where the connectivity features include connectivity features determined from a frequency range of from 35 Hz to 45 Hz divided into two or more sub-bands, and produce an index calculated as a function of one or more of the connectivity features determined from a frequency range of from 35 Hz to 45 Hz divided into two or more sub-bands with varying contribution to the calculation of the index.

The conditioning, determining and producing steps may be as described in the Methods section herein. For purposes of brevity, details regarding signal conditioning, frequency domain feature determination, connectivity feature determination, index production, and other features/elements described in the Methods section of the present disclosure are incorporated but not reiterated herein. In some embodiments, the instructions, when executed by one or more processors, cause the one or more processors to perform any of the methods described in the Methods section herein.

In certain aspects, the instructions, when executed by one or more processors, cause the one or more processors to produce the index calculated as a function of from 5 to 20 total features. In some embodiments, the instructions, when executed by one or more processors, cause the one or more processors to produce the index based on a linear combination of the connectivity features. In certain aspects, the sub-bands are defined with a frequency resolution of from 0.2 Hz to 5 Hz, e.g., 0.5 Hz.

In some embodiments, the instructions, when executed by one or more processors, further cause the one or more processors to generate a report that includes the index. The instructions, when executed by one or more processors, may cause the one or more processors to display the report on a display device (e.g., a display of a desktop computer, laptop computer, television, tablet computer, smartphone, or the like), a printout, or both. The report may include the index displayed graphically in context with data from a database of individuals having dementia and a database of individuals not having dementia. The report may include the index displayed graphically in context with data from a database of individuals having a particular type of dementia and a database of individuals not having dementia. In certain aspects, the report includes the index displayed graphically in context with data from a database of individuals having a first type of dementia and a database of individuals having a second type of dementia. In some embodiments, the first and second types of dementia are selected from the group consisting of: a Lewy Body Dementia, Dementia with Lewy Bodies, Parkinson's Disease Dementia, Alzheimer's Disease Dementia, Frontal Lobe Dementia, and Vascular Dementia. In one example, the first type of dementia is a Lewy Body Dementia and the second type of dementia is Alzheimer's Disease Dementia.

In certain aspects, the instructions, when executed by one or more processors, further cause the one or more processors to diagnose the individual as having dementia based at least in part on the index. The dementia may be dementia generally, or a particular type of dementia selected from the group consisting of: a Lewy Body Dementia, Dementia with Lewy Bodies, Parkinson's Disease Dementia, Alzheimer's Disease Dementia, Frontal Lobe Dementia, and Vascular Dementia. In some embodiments, the instructions, when executed by one or more processors, cause the one or more processors to provide a differential dementia diagnosis. In certain aspects, the instructions, when executed by one or more processors, cause the one or more processors to diagnose the individual as having dementia based on the index and one or more of a neuropsychiatric assessment of the individual, imaging of the individual's brain, and analysis of a biomarker present in a body fluid of the individual. In some embodiments, the instructions, when executed by one or more processors, cause the one or more processors to display via a display a prompt to a medical practitioner to perform one or more of a neuropsychiatric assessment of the individual, imaging of the individual's brain, and analysis of a biomarker present in a body fluid of the individual. In certain aspects, the instructions, when executed by one or more processors, further cause the one or more processors to recommend a treatment for the individual based on the diagnosis.

In some embodiments, the instructions, when executed by one or more processors, further cause the one or more processors to stage the individual's dementia based at least in part on the index. In certain aspects, the instructions, when executed by one or more processors, further cause the one or more processors to assess the progression of the individual's dementia based at least in part on the index. The progression of the individual's dementia may be assessed based at least in part on the index and one or more prior indexes produced for the individual.

Also provided is a non-transitory computer readable medium that includes instructions for producing an index, where the instructions, when executed by one or more processors, cause the one or more processors to condition EEG signals present in an EEG recording previously obtained from an individual, determine frequency domain features from the conditioned EEG signals, determine connectivity features from the frequency domain features, wherein the connectivity features comprise connectivity features determined from a frequency range of from 35 Hz to 45 Hz divided into two or more sub-bands, produce an index calculated as a function of one or more of the connectivity features determined from a frequency range of from 35 Hz to 45 Hz divided into two or more sub-bands with varying contribution to the calculation of the index, and predict the onset of dementia in the individual based at least in part on the index.

Any of the non-transitory computer readable media of the present disclosure may include instructions which, when executed by one or more processors, cause the one or more processors to collect EEG signals from the individual to obtain the EEG recording.

Instructions can be coded onto a non-transitory computer-readable medium in the form of “programming”, where the term “computer-readable medium” as used herein refers to any non-transitory storage or transmission medium that participates in providing instructions and/or data to a computer for execution and/or processing. Examples of storage media include a hard disk, optical disk, magneto-optical disk, CD-ROM, CD-R, magnetic tape, non-volatile memory card, ROM, DVD-ROM, Blue-ray disk, solid state disk, network attached storage (NAS), etc., whether or not such devices are internal or external to the computer. A file containing information can be “stored” on computer readable medium, where “storing” means recording information such that it is accessible and retrievable at a later date by a computer.

The instructions may be in the form of programming that is written in one or more of any number of computer programming languages. Such languages include, for example, Java (Sun Microsystems, Inc., Santa Clara, Calif.), Visual Basic (Microsoft Corp., Redmond, Wash.), and C++ (AT&T Corp., Bedminster, N.J.), as well as many others.

The present disclosure also provides computer devices. The computer devices include one or more processors and any of the non-transitory computer readable media of the present disclosure. Accordingly, in some embodiments, the computer devices are capable of performing any of the methods described in the Methods section herein.

In certain aspects, a computer device of the present disclosure is a local computer device. In some embodiments, the computer device is a remote computer device (e.g., a remote server), meaning that the instructions are executed on a computer device different from a local computer device and/or the instructions are downloadable from the remote computer device to a local computer device, e.g., for execution on the local computer device. In some embodiments, the instructions constitute a web-based application stored on a remote server.

Notwithstanding the appended claims, the present disclosure is also defined by the following embodiments:

1. A computer-implemented method for producing an index, comprising:
conditioning, using one or more processors, electroencephalographic (EEG) signals present in an EEG recording previously obtained from an individual having dementia;
determining, using the one or more processors, frequency domain features from the conditioned EEG signals;
determining, using the one or more processors, connectivity features from the frequency domain features, wherein the connectivity features comprise connectivity features determined from a frequency range of from 35 Hz to 45 Hz divided into two or more sub-bands; and
producing, using the one or more processors, an index calculated at least in part as a function of one or more of the connectivity features determined from a frequency range of from 35 Hz to 45 Hz divided into two or more sub-bands with varying contribution to the calculation of the index.

2. The method according to embodiment 1, wherein the index is calculated as a function of from 5 to 20 connectivity features.

3. The method according to embodiment 1 or embodiment 2, wherein the index is based on a linear combination of the connectivity features.

4. The method according to any one of embodiments 1 to 3, wherein the sub-bands are defined with a frequency resolution of from 0.2 Hz to 5 Hz.

5. The method according to any one of embodiments 1 to 4, wherein, in addition to one or more of the connectivity features, the index is calculated as a function of one or more of the frequency domain features.

6. The method according to embodiment 5, wherein one or more of the frequency domain features are from a frequency range of from 35 Hz to 45 Hz divided into two or more sub-bands with varying contribution to the calculation of the index.

7. The method according to any one of embodiments 1 to 6, wherein the index is further calculated using the sex of the individual as a feature.

8. The method according to any one of embodiments 1 to 7, wherein the index is further calculated using the age of the individual as a feature.

9. The method according to any one of embodiments 1 to 8, comprising harmonizing the frequency domain features based on the type of EEG recording equipment used to obtain the EEG recording.

10. The method according to embodiment 9, wherein the harmonizing is based on calibration response data for the type of EEG recording equipment used to obtain the EEG recording.

11. A computer-implemented method for producing an index, comprising:
conditioning, using one or more processors, electroencephalographic (EEG) signals present in an EEG recording previously obtained from an individual having dementia;
determining, using the one or more processors, frequency domain features from the conditioned EEG signals;
determining, using the one or more processors, connectivity features from the frequency domain features; and
producing, using the one or more processors, an index calculated at least in part as a function of one or more of the connectivity features, and wherein the index is further calculated as a function of the age of the individual, the sex of the individual, or both.

12. The method according to embodiment 11, further comprising harmonizing the frequency domain features based on the type of EEG recording equipment used to obtain the EEG recording.

13. The method according to embodiment 11 or embodiment 12, wherein the connectivity features comprise connectivity features determined from a frequency range of from 35 Hz to 45 Hz divided into two or more sub-bands.

14. A computer-implemented method for producing an index, comprising:
conditioning, using one or more processors, electroencephalographic (EEG) signals present in an EEG recording previously obtained from an individual having dementia;
determining, using the one or more processors, frequency domain features from the conditioned EEG signals;
determining, using the one or more processors, connectivity features from the frequency domain features;
harmonizing, using the one or more processors, the frequency domain features based on the type of EEG recording equipment used to obtain the EEG recording; and
producing, using the one or more processors, an index calculated at least in part as a function of one or more of the connectivity features and the harmonized frequency domain features.

15. The method according to embodiment 14, wherein one or more of the connectivity features are determined from the harmonized frequency domain features.

16. The method according to embodiment 14 or embodiment 15, wherein the harmonizing is based on calibration response data for the type of EEG recording equipment used to obtain the EEG recording.

17. The method according to any one of embodiments 14 to 16, wherein the index is further calculated as a function of the age of the individual, the sex of the individual, or both.

18. The method according to any one of embodiments 14 to 17, wherein the connectivity features comprise connectivity features determined from a frequency range of from 35 Hz to 45 Hz divided into two or more sub-bands.

19. The method according to any one of embodiments 1 to 18, further comprising generating a report comprising the index.

20. The method according to embodiment 19, wherein generating a report comprises displaying the index on a display or printout.

21. The method according to embodiment 20, wherein the index is displayed graphically in context with data from a database of individuals having dementia and a database of individuals not having dementia.

22. The method according to embodiment 20, wherein the index is displayed graphically in context with data from a database of individuals having a particular type of dementia and a database of individuals not having dementia.

23. The method according to embodiment 20, wherein the index is displayed graphically in context with data from a database of individuals having a first type of dementia and a database of individuals having a second type of dementia.

24. The method according to embodiment 23, wherein the first and second types of dementia are selected from the group consisting of: a Lewy Body Dementia, Dementia with Lewy Bodies, Parkinson's Disease Dementia, Alzheimer's Disease Dementia, Frontal Lobe Dementia, and Vascular Dementia.

25. The method according to embodiment 23, wherein the first type of dementia is a Lewy Body Dementia and the second type of dementia is Alzheimer's Disease Dementia.

26. The method according to any one of embodiments 1 to 25, further comprising displaying via a display a suggested diagnosis of dementia based at least in part on the index.

27. The method according to embodiment 26, wherein the suggested diagnosis is of a particular type of dementia.

28. The method according to embodiment 27, wherein the particular type of dementia is selected from the group consisting of: a Lewy Body Dementia, Dementia with Lewy Bodies, Parkinson's Disease Dementia, Alzheimer's Disease Dementia, Frontal Lobe Dementia, and Vascular Dementia.

29. The method according to any one of embodiments 1 to 28, further comprising diagnosing the individual as having dementia based at least in part on the index.

30. The method according to embodiment 29, wherein the diagnosing comprises diagnosing the individual as having a particular type of dementia.

31. The method according to embodiment 30, wherein the individual is diagnosed as having a particular type of dementia selected from the group consisting of: a Lewy Body Dementia, Dementia with Lewy Bodies, Parkinson's Disease Dementia, Alzheimer's Disease Dementia, Frontal Lobe Dementia, and Vascular Dementia.

32. The method according to any one of embodiments 29 to 31, wherein the diagnosing comprises providing a differential dementia diagnosis.

33. The method according to any one of embodiments 29 to 32, wherein the diagnosing is based on the index and one or more of a neuropsychiatric assessment of the individual, imaging of the individual's brain, and analysis of a biomarker present in a body fluid of the individual.

34. The method according to any one of embodiments 29 to 33, further comprising displaying via a display a prompt to perform one or more of a neuropsychiatric assessment of the individual, imaging of the individual's brain, and analysis of a biomarker present in a body fluid of the individual.

35. The method according to any one of embodiments 29 to 34, further comprising recommending a dementia treatment for the individual based on the diagnosis.

36. The method according to any one of embodiments 29 to 35, further comprising treating the individual's dementia based on the diagnosis.

37. The method according to any one of embodiments 1 to 36, further comprising staging the individual's dementia based at least in part on the index.

38. The method according to any one of embodiments 1 to 37, further comprising assessing the progression of the individual's dementia based at least in part on the index.

39. The method according to any one of embodiments 1 to 38, wherein the method is performed iteratively to monitor progression of the individual's dementia.

40. The method according to any one of embodiments 1 to 39, wherein the individual had already been diagnosed as having dementia when the EEG recording was previously obtained.

41. A computer-implemented method for producing an index, comprising:
conditioning, using one or more processors, electroencephalographic (EEG) signals present in an EEG recording previously obtained from an individual;
determining, using the one or more processors, frequency domain features from the conditioned EEG signals;
determining, using the one or more processors, connectivity features from the frequency domain features, wherein the connectivity features comprise connectivity features determined from a frequency range of from 35 Hz to 45 Hz divided into two or more sub-bands;
producing, using the one or more processors, an index calculated at least in part as a function of one or more of the connectivity features determined from a frequency range of from 35 Hz to 45 Hz divided into two or more sub-bands with varying contribution to the calculation of the index; and
predicting the onset of dementia in the individual based at least in part on the index.

42. The method according to any one of embodiments 1 to 41, further comprising, prior to the conditioning, collecting EEG signals from the individual to obtain the EEG recording.

43. A non-transitory computer readable medium comprising instructions, when executed by one or more processors, cause the one or more processors to perform the method according to any one of embodiments 1 to 42.

44. A non-transitory computer readable medium comprising instructions for producing an index, wherein the instructions, when executed by one or more processors, cause the one or more processors to:
condition electroencephalographic (EEG) signals present in an EEG recording previously obtained from an individual having dementia;
determine frequency domain features from the conditioned EEG signals;
determine connectivity features from the frequency domain features, wherein the connectivity features comprise connectivity features determined from a frequency range of from 35 Hz to 45 Hz divided into two or more sub-bands; and
produce an index calculated at least in part as a function of one or more of the connectivity features determined from a frequency range of from 35 Hz to 45 Hz divided into two or more sub-bands with varying contribution to the calculation of the index.

45. The non-transitory computer readable medium of embodiment 44, wherein the instructions, when executed by one or more processors, cause the one or more processors to produce the index calculated as a function of from 5 to 20 connectivity features.

46. The non-transitory computer readable medium of embodiment 44 or embodiment 45, wherein the instructions, when executed by one or more processors, cause the one or more processors to produce the index based on a linear combination of the connectivity features.

47. The non-transitory computer readable medium of any one of embodiments 44 to 46, wherein the sub-bands are defined with a frequency resolution of from 0.2 Hz to 5 Hz.

48. The non-transitory computer readable medium of any one of embodiments 44 to 47, wherein the instructions, when executed by one or more processors, cause the one or more processors to calculate the index as a function of one or more of the frequency domain features in addition to one or more of the connectivity features.

49. The non-transitory computer readable medium of embodiment 48, wherein one or more of the frequency domain features are from a frequency range of from 35 Hz to 45 Hz divided into two or more sub-bands with varying contribution to the calculation of the index.

50. The non-transitory computer readable medium of any one of embodiments 44 to 49, wherein the instructions, when executed by one or more processors, cause the one or more processors to calculate the index using the sex of the individual as a feature.

51. The non-transitory computer readable medium of any one of embodiments 44 to 50, wherein the instructions, when executed by one or more processors, cause the one or more processors to calculate the index using the age of the individual as a feature.

52. The non-transitory computer readable medium of any one of embodiments 44 to 51, wherein the instructions, when executed by one or more processors, cause the one or more processors to harmonize the frequency domain features based on the type of EEG recording equipment used to obtain the EEG recording.

53. The non-transitory computer readable medium of any one of embodiments 44 to 52, wherein the instructions, when executed by one or more processors, further cause the one or more processors to generate a report comprising the index.

54. The non-transitory computer readable medium of embodiment 53, wherein the instructions, when executed by one or more processors, cause the one or more processors to display the report on a display device, a printout, or both.

55. The non-transitory computer readable medium of embodiment 53 or embodiment 54, wherein the report comprises the index displayed graphically in context with data from a database of individuals having dementia and a database of individuals not having dementia.

56. The non-transitory computer readable medium of embodiment 55, wherein the report comprises the index displayed graphically in context with data from a database of individuals having a particular type of dementia and a database of individuals not having dementia.

57. The non-transitory computer readable medium of any one of embodiments 53 to 55, wherein the report comprises the index displayed graphically in context with data from a database of individuals having a first type of dementia and a database of individuals having a second type of dementia.

58. The non-transitory computer readable medium of embodiment 57, wherein the first and second types of dementia are selected from the group consisting of: a Lewy Body Dementia, Dementia with Lewy Bodies, Parkinson's Disease Dementia, Alzheimer's Disease Dementia, Frontal Lobe Dementia, and Vascular Dementia.

59. The non-transitory computer readable medium of embodiment 57, wherein the first type of dementia is a Lewy Body Dementia and the second type of dementia is Alzheimer's Disease Dementia.

60. The non-transitory computer readable medium of any one of embodiments 44 to 59, wherein the instructions, when executed by one or more processors, further cause the one or more processors to diagnose the individual as having dementia based at least in part on the index.

61. The non-transitory computer readable medium of embodiment 60, wherein the instructions, when executed by one or more processors, cause the one or more processors to diagnose the individual as having a type of dementia selected from the group consisting of: a Lewy Body Dementia, Dementia with Lewy Bodies, Parkinson's Disease Dementia, Alzheimer's Disease Dementia, Frontal Lobe Dementia, and Vascular Dementia.

62. The non-transitory computer readable medium of embodiment 60 or 61, wherein the instructions, when executed by one or more processors, cause the one or more processors to provide a differential dementia diagnosis.

63. The non-transitory computer readable medium of any one of embodiments 60 to 62, wherein the instructions, when executed by one or more processors, cause the one or more processors to diagnose the individual as having dementia based on the index and one or more of a neuropsychiatric assessment of the individual, imaging of the individual's brain, and analysis of a biomarker present in a body fluid of the individual.

64. The non-transitory computer readable medium of any one of embodiments 60 to 63, wherein the instructions, when executed by one or more processors, cause the one or more processors to display via a display a prompt to a medical practitioner to perform one or more of a neuropsychiatric assessment of the individual, imaging of the individual's brain, and analysis of a biomarker present in a body fluid of the individual.

65. The non-transitory computer readable medium of any one of embodiments 44 to 64, wherein the instructions, when executed by one or more processors, further cause the one or more processors to recommend a treatment for the individual based on the diagnosis.

66. The non-transitory computer readable medium of any one of embodiments 44 to 65, wherein the instructions, when executed by one or more processors, further cause the one or more processors to stage the individual's dementia based at least in part on the index.

67. The non-transitory computer readable medium of any one of embodiments 44 to 66, wherein the instructions, when executed by one or more processors, further cause the one or more processors to assess the progression of the individual's dementia based at least in part on the index.

68. The non-transitory computer readable medium of embodiment 67, wherein the progression of the individual's dementia is assessed based at least in part on the index and one or more prior indexes produced for the individual.

69. A non-transitory computer readable medium comprising instructions for producing an index, wherein the instructions, when executed by one or more processors, cause the one or more processors to:
condition EEG signals present in an EEG recording previously obtained from an individual;
determine frequency domain features from the conditioned EEG signals;
determine connectivity features from the frequency domain features, wherein the connectivity features comprise connectivity features determined from a frequency range of from 35 Hz to 45 Hz divided into two or more sub-bands;
produce an index calculated at least in part as a function of one or more of the connectivity features determined from a frequency range of from 35 Hz to 45 Hz divided into two or more sub-bands with varying contribution to the calculation of the index; and
predict the onset of dementia in the individual based at least in part on the index.

70. A non-transitory computer readable medium comprising instructions for producing an index, wherein the instructions, when executed by one or more processors, cause the one or more processors to:
condition EEG signals present in an EEG recording previously obtained from an individual having dementia;
determine frequency domain features from the conditioned EEG signals;
determine connectivity features from the frequency domain features; and
produce an index calculated at least in part as a function of one or more of the connectivity features, and wherein the index is further calculated as a function of the age of the individual, the sex of the individual, or both.

71. A non-transitory computer readable medium comprising instructions for producing an index, wherein the instructions, when executed by one or more processors, cause the one or more processors to:
condition EEG signals present in an EEG recording previously obtained from an individual having dementia;
determine frequency domain features from the conditioned EEG signals;
harmonize the frequency domain features based on the type of EEG recording equipment used to obtain the EEG recording;
determine connectivity features from the frequency domain features; and
produce an index calculated at least in part as a function of one or more of the connectivity features and the harmonized frequency domain features.

72. The non-transitory computer readable medium of any one of embodiments 44 to 71, wherein the instructions, when executed by one or more processors, further cause the one or more processors to collect EEG signals from the individual to obtain the EEG recording.

73. A computer device, comprising:
one or more processors; and
the non-transitory computer readable medium of any one of embodiments 43 to 72.

74. The computer device of embodiment 73, wherein the computer device is a local computer.

75. The computer device of embodiment 73, wherein the computer device is a remote server.

The following examples are offered by way of illustration and not by way of limitation.

### Experimental

### EXAMPLE 1

### Improved Differentiation of Lewy Body Dementias from Alzheimer's Disease Using Higher Frequency Features

I. Introduction

One of the main tasks in the diagnostic work up of cognitive impairment and dementia is to differentiate between the various causes. The current clinical criteria for diagnosis of the most prevalent forms of dementia are of varying accuracy and up to 10% of cases of dementia are difficult to diagnose clinically with reasonable confidence [1].

Alzheimer's disease (AD) is the most common neurodegenerative disease, accounting for more than half of all dementia cases [1, 2]. Biomarkers play an increasingly important role in AD diagnosis [3, 4]. The second most common neurodegenerative disorder causing dementia is the combined and related constellation of Parkinson's Disease Dementia (PDD) and Dementia of Lewy Bodies (DLB), collectively termed Lewy Body Dementias (LBD). The point-prevalence of dementia is roughly 25% in patients with Parkinsons disease. The risk of dementia rises with duration and reaches 50%, 10 years after diagnosis [5]. There is an ongoing debate whether these entities represent two different clinical disorders or if they should be considered as different aspects of one entity [6].

In the 2017 report of the DLB consortium, EEG changes are listed as one of the supportive biomarkers for the diagnosis of the condition [7]. Over the past 30 years many studies have been performed using a wide variety of analysis methods. Some studies have supported EEG as a valid biomarker for DLB and/or PDD while others have not shown useful performance. In a multi-center study validating EEG analyzed with Statistical Pattern

Recognition in the diagnostic work up in six Nordic Memory Clinics, the method distinctly separated the DLB/PDD patients from both AD patients, other patients with and without dementia and the controls [8, 9]. A separate separate multi-center cohort study validated a different set of EEG-derived measures [10]. Only these two studies have reported independent validation of pre-defined EEG biomarkers [10, 8] and none investigated the potential of higher frequencies, in the gamma band, to enable improved robustness.

To date, positive results have primarily focused on alpha and delta bands with various measures, and some on measures of theta band activity. In the present study, the inclusion of higher frequencies ranging from 35 to 45 Hz in EEG biomarkers was investigated for its potential impact on diagnostic performance separating DLB/PDD from AD individuals. The present study demonstrates the importance of including higher frequencies than previously contemplated in order to promote robust differentiation of individuals having DLB/PDD from individuals having AD in routine clinical practice.

Diagnostic Criteria

Most important for the diagnosis of DLB are the core clinical features [7]. Fluctuating cognition, visual hallucinations and REM sleep behavior disorder typically occur early and may even precede cognitive decline. The fourth core clinical feature is parkinsonism, where one or more spontaneous cardinal feature may be observed: bradykinesia, rest tremor or rigidity. In addition, the following clinical symptoms are considered supportive of DLB diagnosis: severe sensitivity to antipsychotic agents, postural instability, repeated falls, syncope, severere autonomous dysfunction (e.g. constipa-tion, orthostatic hypotension or urinary incontinence), hyposmia, hallucinations in other modalities, systematized delusions, apathy, anxiety and depression.

While there is a consensus on different biomarkers for Alzheimer's disease [11, 3, 4], search for biologic markers for Parkinson's Disease (PD) and/or Lewy body dementia has not been as successful yet [12], although three indirect markers are regarded as supportive: reduced dopamine transporter uptake in basal ganglia (SPECT/PET), abnormally low uptake of ¹²³iodine-MIBG myocardial scintig-raphy, and REM sleep without atonia confirmation by Polysomnography (PSG) [7].

EEG is a neurophysiological marker of cortical activity and since both AD and DLB are primarily disorders of the cerebral cortex it stands to reason that EEG might contribute to both pathological research and development of diagnostic measures. The main EEG abnormalities in AD have thus been known for a long time: slowing and a decrease in alpha activity with a corresponding increase in theta and delta activities [13]. In an earlier study, this group has presented EEG with statistical patterns (SPR) analysis as a possible marker for AD, separating AD patients from controls with a sensitivity of 86% and a specificity of 87% [14].

Application of EEG in LBD

A recent systematic review by Cromarty et al. details neurophysiological biomarkers for LBD [15], covering EEG, event-related potentials, magnetoencephalography (MEG), blink reflexes, transcranial magnetic stimulation, and other related techniques. In terms of EEG analyses, the review considered several analytical approaches. Grant Total EEG (GTE) score [16] is based on visual review and manual scoring and will not be considered here. In general, methods based on visual inspection tend to have low specificity [15]. The focus of the present study are methods suitable for automated and semi-automated analysis of EEG.

Power Spectra and Frequency Bands

Kai et al. reported increased power density in 15 DLB cases in delta and theta bands but not in 15 AD cases compared to controls [17]. Furthermore, by analyzing the effect of donepezil in both conditions, the difference between treatments with this drug was greater in DLB indicating more cholinergic dysfunction than in AD. Anderson et al in 2008 analyzed data from 20, 64 and 54 individuals with DLB or AD and healthy controls respectively [18]. They found increased variability and increased overall coherence in delta bands and consequently, decreased overall coherence in the alpha bands. However, other studies indicate similar changes are present in AD suggesting this observation may not be specific to LBD, but may apply more broadly to dementia [19]. Administration of cholinesterase inhibitors has been shown to affect resting state EEG and specific changes in the EEG signals to correlate with successful treatment [20]. Dauwan et al. reported that decrease in alpha-1 and beta band activity as well as EEG slowing showed promise as neurophysiological indicators of hallucinations in AD and DLB.

The utility of exact low-resolution brain electromagnetic source tomography (eLoreta) in dementia differential diagnosis has been studied by Babiloni et al. who reported a marked slowing of the individual alpha frequency peak (IAF) was observed in PDD and DLB subjects compared to normal elderly subjects (Nold), and a moderate slowing in AD compared to Nold [21]. In addition, all groups showed lower posterior alpha-2 source activities and higher occipital delta source activities. Importantly, posterior delta and alpha sources showed sufficient discriminatory power to enable good classification accuracy in the range of 85-90% when classifying the dementia groups compared to Nold.

Topography

Standard EEG is typically recorded from 20 electrodes placed at specific locations according to a standard system (IS 10-20). According to Bonanni et al., topographical differences are apparent in the EEG abnormalities when comparing DLB and AD, where abnormalities are typically observed in the posterior regions in DLB, whereas AD patients tend to exhibit changes in temporal areas [22, 23]. Several studies have reported increased posterior slow-wave activity [22, 24, 25, 26, 27] as mentioned in the preceding section in the context of the clinical diagnostic criteria.

Coherence/Connectivity

Coherence measures reflect connectivity between cortical regions, which has been suggested may reflect modulatory effects of cholinergic deficits that are prominent in LBD [28]. In a study of DLB sub- jects exploring coherence between four major regions (left anterior, right anterior, left posterior and right posterior) greater average coherence between all regions was observed in the delta band, while the same was reduced in the alpha band [18]. In a separate study, differences in intra-hemispheric coherence values were observed when comparing DLB to AD subjects [17], with spatial patterns consistent with cholinergic dysfunction [17].

Following on the power spectral study described above in section 1.2.1, the Babiloni group has studied the utility of eLoreta functional lagged linear connectivity (LLC) and reported that inter- and intra-hemispheric LLC sources involving delta sources were abnormally high in AD, but appeared normal in DLB and PDD [29], while intra-hemispheric LLC sources involving alpha were decreased in AD, DLB and PDD.

Dauwan et al. applied phase transfer entropy to measure directed connectivity in DLB and AD groups, assessing the theta, alpha and beta frequency bands [30]. They found that a posterior-anterior phase transfer entropy gradient, observed in controls where occipital channels were driving frontal channels, was largely lost in the alpha band in DLB subjects. The effect was statistically significant.

Applying brain network analysis based on minimum spanning trees, Pereza et al. studied network properties in DLB, AD and healthy control subjects [31]. They found that alpha band MST networks were a good indicator of dementia compared to controls, while beta band phase lag index and domi-nant frequency variability differentiated between DLB and AD subjects.

Background Frequency

Several studies have include the dominant background rhythm frequency by using power spectra. One group reported significant differences in this parameter between PD, PD-MCI and PDD groups [32], although a limited number of subjects were included in the study.

Fluctuations

The current diagnostic criteria [7] note that prominent posterior slow wave activity with fluctuations in the pre-alpha/theta range support DLB diagnosis supported by four published studies. Bonanni et al. described patterns of pre-alpha-dominant frequency, either stable or intermixed with alpha/theta/delta activities in pseudo-periodic patterns [22], which the group later showed to have strong accuracy in differential diagnosis of DLB against AD [10] that may be seen at the MCI stage [33] and is consistent with patterns previously shown to correlate with severity of cognitive fluctuations [34].

Fluctuations in the frequency spectra on a second-by-second basis have been observed in DLB patients and connections drawn with cognitive fluctuations [34, 22], and shown to be influenced by administration of cognition-enhancing medications like acetylcholinesterase inhibitors [17]. Bonanni et al. have described a particularly effective, semi-automatic way to visualize such fluctuations using compressed spectral arrays highlighting dominant frequency and variability over time of dominant frequency [22]. Her group has used this same methodology in further studies [23, 33].

In a recent study, Stylianou et al. confirmed previous findings of EEG slowing in DLB and PDD groups compared with both AD and normal control subjects [35]. They did not observe higher domi-nant frequency variability (DFV) in DLB compared to controls as would have been suspected. How-ever, they observed a correlation between DFV and cognitive fluctuations that are among the core clin-ical features of DLB. Lastly, they reported strong classification performance of fast-theta frequency prevalence, theta power and theta-alpha dominant frequency variability distinguishing between DLB and AD.

Multi-Variate Classifiers

Multi-variate classifiers for DLB/PDD enable significant improvement in DLB specificity over AD [15]. The Mentis Cura research team has published six articles on EEG diagnostic tools that were the precursor to the results described in this document.

Gudmundsson et al. reported learnings on reference settings, duration or recording and relative reliability of different features such as power spectral bands that were observed to be more stable than coherence features [36].

In a pilot study, the ability of principal component analysis (PCA) help to classify

Alzheimer's Disease, Mild Cognitive Impairment and normal controls one from another was reported, including the utilization of a pharmaceutical challenge (scopolamine) to enhance the separation [37]. The PCA was based on 28 features including power spectral bands (as well as various derived parameters such as total power, and ratios), peak alpha frequency, median frequency, entropy, activity, mobility and complexity. In a follow-on paper, the group refined the list to 20 features and added 27 new coher-ence features representing various intra-hemispheric, inter-hemispheric and local connections [14]. Classification accuracy for all possible pairs of seven groups (Normal controls, AD, Vascular Dementia, stable MCI, DLB/PDD, Frontal Lobe Dementia and Depression) were reported with strong performance ranging from 73 to 97% accuracy, the highest being observed for DLB/PDD vs. NRM and DLB/PDD vs. AD very strong at 91%. In all cases the performance was estimated by 10-fold cross-validation and not at the time confirmed with independent data.

Johannsson et al. reported the same methodology applied to pharmacological challenge data (scopolamine) that led to the creation of a multi-variate index calculated from 20 power spectral and coherence features from the electroencephalogram [38]. The index was shown to correlate with cognitive tests and severity of dementia clinical diagnosis. Together with collaborators at Newcastle University, the group reported a multi-variate index based on 20 EEG features and enhanced per-formance when combined with medial temporal lobe atrophy metrics derived from MRI images [39]. Comparable methods were applied to develop a classifier for Attention Deficit Hyperactivity Disorder (ADHD) [40].

A consortium of six clinical research centers has independently reported on the diagnostic performance of Mentis Cura's diagnostic indices confirming strong performance distinguishing dementia (and AD specifically) from normal controls as well as when separating DLB/PDD from other dementia (and AD specifically) [8, 9].

Diagnostic Applications

In a study of Bonanni et al. in 2008 [22], 140 cases of DLB, PDD and AD were followed for two years and the diagnosis was then revised. Diagnosis was not changed in 36/50 DLB cases 35/40 PDD cases and 40/50 AD cases making the final clinical decision more robust. By assessing EEG variability by mean frequency analysis and compressed spectral arrays (CSA) in order to detect changes over time, there was a significant group difference between DLB and AD cases. Of note was that half of PDD patients did not exhibit the changes seen in DLB. In these studies, the stage of dementia was not been taken into consideration. Recently, Bonanni et al showed that individuals with MCI and EEG abnormalities of reduced dominant frequency and increased dominant frequency variability were likely to convert to DLB in the following years [33]. In a recent study, Stylianou et al. were not able to replicate the findings that fluctuations could distinguish between DLB and AD [35].

In a study of Caviness et al. in 2011 [41], electrophysiological findings in incidental Lewy body disease (ILBD) indicated that EEG could be used in the diagnosis of this disorder before onset of dementia. In that study, ILBD was defined post mortem as a disorder without any clinical signs of Lewy body disease but with Lewy bodies in cortex. ILBD is now considered to be the most prevalent Lewy body disorder [42] and to represent a preclinical stage of either Parkinson's disease or DLB [43]. It is stated in their report that the ILBD group (n=10) had significantly lower background rhythm frequency mean than the control group (n=28), but higher than a PD group (n=7).

Clinical Validation

A requirement for medical devices prior to commercial use is that their performance has been validated in appropriately designed studies. For EEG diagnostics, prospective clinical trials with fixed and finalized biomarker designs and cut-off values as well as a pre-specified statistical analysis plan are most appropriate. The history of EEG diagnostic applications reflects how easily one can be misled by apparently positive results that, upon further investigation, cannot be replicated in independent studies [44]. Therefore, information about robust performance for independent cohorts is of great utility for EEG biomarkers and will generally be necessary before such biomarkers are endorsed in clinical guidelines [45].

II. Materials and Methods

Participants

Training Data Set

For optimization of the multi-variate classifiers, data from a previous study [14] was used. The study participants and diagnostic assessments are described in the methods section of the paper, and the characteristics of the groups are described in Table 1. For this work, 237 AD subjects (including those with mixed AD and Vascular Dementia) and 51 DLB/PDD subjects were included. The study was performed according to Good Clinical Practice and all participants signed a written informed consent form prior to participation. The study protocol was accepted by the National Research Ethics Committee in Iceland.

Validation Data Set

The data used for the validation are from an independent study reported by Engedal et al. [8]. The study participants and diagnostic assessments are described in the methods section of the paper, and the characteristics of the groups are described in Table 2. For this analysis, 100 probable AD [46] subjects and 15 DLB/PDD subjects were included. The study was performed according to Good Clinical Practice and all participants signed a written informed consent form prior to participation. The study protocol was accepted by the National Research Ethics Committees in each participating country.

EEG Registration and Signal Processing

The EEG was recorded at rest with eyes closed for 5 minutes and the subjects were alerted if they became visibly drowsy. Electrodes were placed according to the IS 10-20 system with 19 electrodes: Fp1, Fp2, F3, F4, F7, F8, Fz, T3, T4, T5, T6, C3, C4, Cz, P3, P4, Pz, O1 and O2. Recordings were referenced to the average potential and two bipolar electro-oculography channels and one ECG were applied to monitor artefacts. The EEGs were measured using the NicoletOne nEEG Module (Natus Medical Inc., Pleasanton, Calif., USA).

Subsequent analysis was done in the Matlab environment (MathWorks, Natick, Mass., USA) as previously described [14, 8, 39]. Digital signal processing was applied to eliminate noise, fast fourier transform to calculate the power spectra for each channel for each subject, and coherence measures determined in order to estimate connectivity leading to 1,900 features describing each EEG recording.

Classifier Development

Using Statistical pattern recognition as previously described [14, 8, 39], a large number of classifiers were created based on the training data set. Briefly, a genetic algorithm was applied to optimize the selection of 10 features (out of 1,900) to achieve the strongest performance classifying the DLB/PDD subjects vs. the AD subjects. The optimal performance for each set of features was determined using Support Vector Machines (SVM) and modified 5-fold cross-validation. The optimization was run for 50 iterations, which generated over 7,000 classifiers. In each case examined here, the performance had reached a plateau after approximately 30 iterations.

Two distinct sets of classifiers were produced. The first set using the frequency range of 0.5 to 35 Hz (“LP 35 Hz”) and the second set using the frequency range 0.5 to 45 Hz (“LP 45 Hz”). Both had an equal number of features (i.e., 1,900) since in each case 10 features are used to represent the frequency dimension across the frequency-domain transform for each channel (19 channels) as well as the coherence measures for each channel pair (171 pairs).

Performance Analysis

The performance of different sets of classifiers was analyzed using the R statistical computing environment [47, 48, 49] and applying the Welch two sample t-test to test for true difference in means.

III. Results

Reliability of Diagnostic Performance for Independent Data

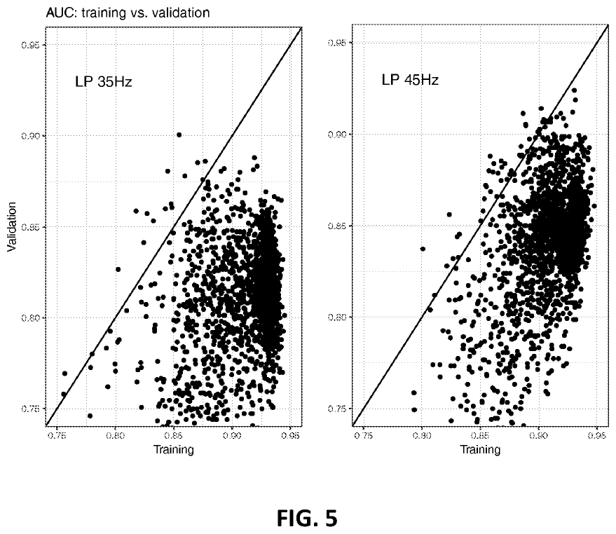

The diagnostic performance assessed on the training set was compared with that assessed on the independent validation set as shown in FIG. 5. For this comparison, only classifiers identified after the optimization process had reached a plateau were considered. Specifically, 2500 classifiers that were produced after 30 iterations of the genetic algorithm evolution had been completed. FIG. 5 shows scatter plots contrasting the training performance (x-axis) with the validation performance (y-axis), with the two different sets, allowing 0.5-35 Hz (LP-35 Hz) and 0.5-45 Hz (LP-45 Hz), shown in the left and right panels of FIG. 5, respectively.

While both LP-35 Hz and LP-45 Hz populations achieve similar performance on the training set, there is a marked difference for the independent validation set performance. Many of the LP-45 Hz classifiers reach higher performance on the independent validation set compared to the LP-35 Hz classifiers. This is especially true when looking at those classifiers that perform well on the training set.

Including Gamma Frequencies Increases Reliability

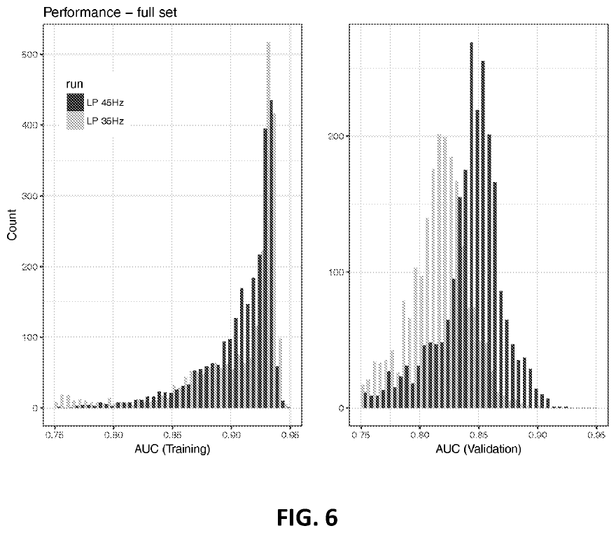

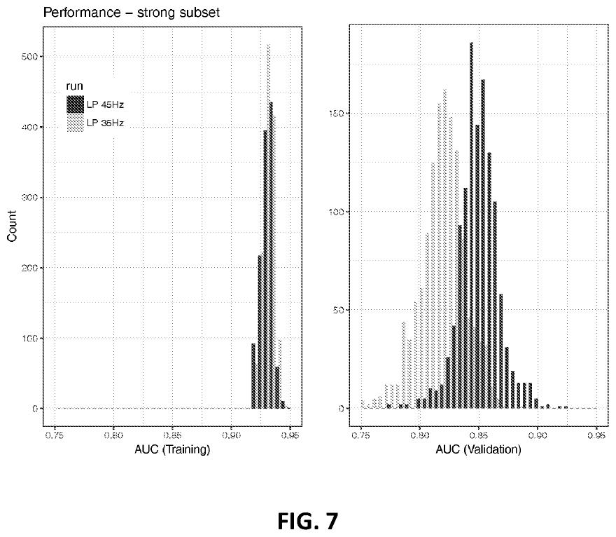

Observing the distribution of AUC values based on the training data sets there is negligible difference in the peak value when comparing LP-45 Hz to LP-35 Hz as reflected in the left panel of FIG. 6. However, when the distribution of AUC values for the same classifiers applied to an independent validation set is assessed, separation of the distributions is significant with difference in means of 0.053 (95% confidence interval: 0.049-0.057). This is shown in the right panel of FIG. 6. FIG. 7 shows analogous distributions where only high performing classifiers are considered—those classifiers that show AUC>0.92 when applied to the training set. In this case there is no significant difference between the means of AUC values for LP-35 Hz compared to LP-45 Hz when applied to the training set (p=0.23). Both are estimated to be 0.93 reflected in the histogram shown in the left panel of FIG. 7. However, there is a highly significant difference in means (p<2.2 10-16) of 0.851 and 0.817, for LP-35 Hz and LP-45 Hz, respectively.

IV. Discussion

The robustness of EEG diagnostic measures when applied to indepedent data is crucial for clinical utility of the technology. Previous studies have focused on the alpha, delta and theta bands. Shown here is that including the gamma band in the optimization of multi-variate classifiers confers a significant improvement in robustness. This is reflected in the results above showing that classifiers produced using frequencies only up to 35 Hz showed similar performance on the training set when compared to those produced using frequencies up to 45 Hz (FIG. 6, left panel), but significantly lower performance when applied to an independent training set (FIG. 6, right panel). The difference becomes even more clear when focusing only on those classifiers that show performance of AUC>0.92 on the training set (FIG. 7). These results demonstrate that inclusion of gamma frequencies has a positive impact on diagnostic performance of EEG biomarkers for future use in routine clinical practice.

V. References

[1] D. S. Knopman, S. T. DeKosky, J. Cummings, H. Chui, J. Corey-Bloom, N. Relkin, G. Small, B. Miller, and J. Stevens, “Practice parameter: diagnosis of dementia (an evidence-based re-view): report of the quality standards subcommittee of the american academy of neurology,” Neurology, vol. 56, no. 9, pp. 1143-1153, 2001.

[2] A. Association et al., “2018 alzheimer's disease facts and figures,” Alzheimer's & Dementia, vol. 14, no. 3, pp. 367-429, 2018.

[3] G. M. McKhann, D. S. Knopman, H. Chertkow, B. T. Hyman, C. R. Jack Jr, C. H. Kawas, W. E. Klunk, W. J. Koroshetz, J. J. Manly, R. Mayeux, et al., “The diagnosis of dementia due to alzheimer's disease: Recommendations from the national institute on aging-alzheimer's asso-ciation workgroups on diagnostic guidelines for alzheimer's disease,” Alzheimer's & dementia, vol. 7, no. 3, pp. 263-269, 2011.

[4] B. Dubois, H. H. Feldman, C. Jacova, H. Hampel, J. L. Molinuevo, K. Blennow, S. T. DeKosky, S. Gauthier, D. Selkoe, R. Bateman, et al., “Advancing research diagnostic criteria for alzheimer's disease: the iwg-2 criteria,” The Lancet Neurology, vol. 13, no. 6, pp. 614-629, 2014.

[5] D. Aarsland, K. Andersen, J. P. Larsen, and A. Lolk, “Prevalence and characteristics of demen-tia in parkinson disease: an 8-year prospective study,” Archives of neurology, vol. 60, no. 3, pp. 387-392, 2003.

[6] D. Aarsland, E. Londos, and C. Ballard, “Parkinson's disease dementia and dementia with lewy bodies: different aspects of one entity,” International psychogeriatrics, vol. 21, no. 2, pp. 216-219, 2009.

[7] I. G. McKeith, B. F. Boeve, D. W. Dickson, G. Halliday, J.-P. Taylor, D. Weintraub, D. Aarsland, J. Galvin, J. Attems, C. G. Ballard, et al., “Diagnosis and management of dementia with lewy bodies: Fourth consensus report of the dlb consortium,” Neurology, vol. 89, no. 1, pp.

88-100, 2017.

[8] K. Engedal, J. Snaedal, P. Hoegh, V. Jelic, B. B. Andersen, M. Naik, L.-O. Wahlund, and A.-R. Oeksengaard, “Quantitative eeg applying the statistical recognition pattern method: a useful tool in dementia diagnostic workup,” Dementia and geriatric cognitive disorders, vol. 40, no. 1-2, pp. 1-12, 2015.

[9] D. Ferreira, V. Jelic, L. Cavallin, A.-R. Oeksengaard, J. Snaedal, P. Hogh, B. B. Andersen, M. Naik, K. Engedal, E. Westman, and L.-O. Wahlund, “Electroencephalography Is a Good Complement to Currently Established Dementia Biomarkers,” DEMENTIA AND GERIATRIC COGNITIVE DISORDERS, vol. 42, no. 1-2, pp. 80-92, 2016.

[10] L. Bonanni, R. Franciotti, F. Nobili, M. G. Kramberger, J.-P. Taylor, S. Garcia-Ptacek, N. W. Falasca, F. Fama, R. Cromarty, M. Onofrj, D. Aarsland, and E.-D. S. Grp, “EEG Markers of Dementia with Lewy Bodies: A Multicenter Cohort Study,” JOURNAL OF ALZHEIMERS DISEASE, vol. 54, no. 4, pp. 1649-1657, 2016.

[11] B. Dubois, H. H. Feldman, C. Jacova, J. L. Cummings, S. T. DeKosky, P. Barberger-Gateau, A. Delacourte, G. Frisoni, N. C. Fox, D. Galasko, et al., “Revising the definition of alzheimer's disease: a new lexicon,” The Lancet Neurology, vol. 9, no. 11, pp. 1118-1127, 2010.

[12] D. P. Breen, A. W. Michell, and R. A. Barker, “Parkinson's disease—the continuing search for biomarkers,” Clinical chemistry and laboratory medicine, vol. 49, no. 3, pp. 393-401, 2011.

[13] J. Jeong, “Eeg dynamics in patients with alzheimer's disease,” Clinical neurophysiology, vol. 115, no. 7, pp. 1490-1505, 2004.

[14] J. Snaedal, G. H. Johannesson, T. E. Gudmundsson, N. P. Blin, A. L. Emilsdottir, B. Einarsson, and K. Johnsen, “Diagnostic accuracy of statistical pattern recognition of electroencephalogram registration in evaluation of cognitive impairment and dementia,” Dementia and geriatric cog-nitive disorders, vol. 34, no. 1, pp. 51-60, 2012.

[15] R. A. Cromarty, G. J. Elder, S. Graziadio, M. Baker, L. Bonanni, M. Onofrj, J. T. O'Brien, and J.-P. Taylor, “Neurophysiological biomarkers for Lewy body dementias,” CLINICAL NEURO-PHYSIOLOGY, vol. 127, pp. 349-359, January 2016.

[16] J. Claus, R. Strijers, E. Jonkman, B. de Visser, C. Jonker, G. Walstra, P. Scheltens, and W. van Gool, “The diagnostic value of electroencephalography in mild senile Alzheimer' s disease,” CLINICAL NEUROPHYSIOLOGY, vol. 110, pp. 825-832, May 1999.

[17] T. Kai, Y. Asai, K. Sakuma, T. Koeda, and K. Nakashima, “Quantitative electroencephalogram analysis in dementia with lewy bodies and alzheimer's disease,” Journal of the neurological sciences, vol. 237, no. 1-2, pp. 89-95, 2005.

[18] M. Andersson, O. Hansson, L. Minthon, I. Rose'n, and E. Londos, “Electroencephalogram vari-ability in dementia with lewy bodies, alzheimer's disease and controls,” Dementia and geriatric cognitive disorders, vol. 26, no. 3, pp. 284-290, 2008.

[19] C. E. Jackson and P. J. Snyder, “Electroencephalography and event-related potentials as biomarkers of mild cognitive impairment and mild Alzheimer's disease,” ALZHEIMERS & DEMENTIA, vol. 4, pp. S137-S143, January 2008. Leon Thal Symposium on the Prevention of Dementia (LTS 07), Las Vegas, Nev., Dec. 2-4, 2007.

[20] C. Babiloni, C. Del Percio, R. Bordet, J.-L. Bourriez, M. Bentivoglio, P. Payoux, P. Derambure, S. Dix, F. Infarinato, R. Lizio, et al., “Effects of acetylcholinesterase inhibitors and meman-tine on resting-state electroencephalographic rhythms in alzheimer's disease patients,” Clinical Neurophysiology, vol. 124, no. 5, pp. 837-850, 2013.

[21] C. Babiloni, C. Del Percio, R. Lizio, G. Noce, S. Cordone, S. Lopez, A. Soricelli, R. Ferri, M. T. Pascarelli, F. Nobili, et al., “Abnormalities of cortical neural synchronization mechanisms in patients with dementia due to alzheimer's and lewy body diseases: an eeg study,” Neurobiology of aging, vol. 55, pp. 143-158, 2017.

[22] L. Bonanni, A. Thomas, P. Tiraboschi, B. Perfetti, S. Varanese, and M. Onofrj, “Eeg com-parisons in early alzheimer's disease, dementia with lewy bodies and parkinson's disease with dementia patients with a 2-year follow-up,” Brain, vol. 131, no. 3, pp. 690-705, 2008.

[23] L. Bonanni, R. Franciotti, V. Onofrj, F. Anzellotti, E. Mancino, D. Monaco, F. Gambi, L. Man- zoli, A. Thomas, and M. Onofrj, “Revisiting P300 cognitive studies for dementia diagnosis: Early dementia with Lewy bodies (DLB) and Alzheimer disease (AD),” NEUROPHYSIOLO-GIE CLINIQUE-CLINICAL NEUROPHYSIOLOGY, vol. 40, pp. 255-265, November-December 2010.

[24] R. Briel, I. McKeith, W. Barker, Y. Hewitt, R. Perry, P. Ince, and A. Fairbairn, “EEG findings in dementia with Lewy bodies and Alzheimer's disease,” JOURNAL OF NEUROLOGY NEURO- SURGERY AND PSYCHIATRY, vol. 66, pp. 401-403, March 1999.

[25] S. Calzetti, E. Bortone, A. Negrotti, L. Zinno, and D. Mancia, “Frontal intermittent rhythmic delta activity (FIRDA) in patients with dementia with Lewy bodies: a diagnostic tool?,” NEU-ROLOGICAL SCIENCES, vol. 23, pp. S65-S66, SEP 2002. 28th National Congress on Exper-imental Models and Clinical Correclations in Extrapyramidal Diseases and Dementia, ALBA, ITALY, Nov. 7-9, 2001.

[26] J. L. Fernandez-Torre, J. Figols, I. Alonso, C. Leno, M. Martinez-Martinez, R. Carpizo, and M. Altable, “Detailed electroencephalographic long-term follow-up study in Lewy body demen-tia with periodic sharp wave complexes,” JOURNAL OF NEUROLOGY, vol. 254, pp. 384-387, March 2007.

[27] G. Roks, E. Korf, W. Van der Flier, P. Scheltens, and C. Stam, “The use of eeg in the diagnosis of dementia with lewy bodies,” Journal of Neurology, Neurosurgery & Psychiatry, 2007.

[28] G. Adler, S. Brassen, and A. Jajcevic, “EEG coherence in Alzheimer's dementia,” JOURNAL OF NEURAL TRANSMISSION, vol. 110, pp. 1051-1058, September 2003.

[29] C. Babiloni, C. Del Percio, R. Lizio, G. Noce, S. Lopez, A. Soricelli, R. Ferri, F. Nobili, D. Ar-naldi, F. Fama', et al., “Abnormalities of resting-state functional cortical connectivity in patients with dementia due to alzheimer's and lewy body diseases: an eeg study,” Neurobiology of aging, vol. 65, pp. 18-40, 2018.

[30] M. Dauwan, E. van Dellen, L. van Boxtel, E. C. W. van Straaten, H. de Waal, A. W. Lemstra, A. Gouwa, W. M. van der Flier, P. Scheltens, I. E. Sommer, and C. J. Stam, “EEG-directed connectivity from posterior brain regions is decreased in dementia with Lewy bodies: a compar-ison with Alzheimer's disease and controls,” NEUROBIOLOGY OF AGING, vol. 41, pp. 122-129, May 2016.

[31] L. R. Peraza, R. Cromarty, X. Kobeleva, M. J. Firbank, A. Killen, S. Graziadio, A. J. Thomas, J. T. O'Brien, and J.-P. Taylor, “Electroencephalographic derived network differences in Lewy body dementia compared to Alzheimer's disease patients,” SCIENTIFIC REPORTS, vol. 8, Mar. 15, 2018.

[32] J. N. Caviness, J. G. Hentz, V. G. Evidente, E. Driver-Dunckley, J. Samanta, P. Mahant, D. J. Connor, M. N. Sabbagh, H. A. Shill, and C. H. Adler, “Both early and late cognitive dysfunction affects the electroencephalogram in Parkinson's disease,” PARKINSONISM & RELATED DISORDERS, vol. 13, no. 6, pp. 348-354, 2007.

[33] L. Bonanni, B. Perfetti, S. Bifolchetti, J.-P. Taylor, R. Franciotti, L. Parnetti, A. Thomas, and M. Onofrj, “Quantitative electroencephalogram utility in predicting conversion of mild cognitive impairment to dementia with lewy bodies,” Neurobiology of aging, vol. 36, no. 1, pp. 434-445, 2015.

[34] M. Walker, G. Ayre, J. Cummings, K. Wesnes, I. McKeith, J. O'Brien, and C. Ballard, “Quan-tifying fluctuation in dementia with Lewy bodies, Alzheimer's disease, and vascular dementia,” NEUROLOGY, vol. 54, pp. 1616-1624, Apr. 25, 2000.

[35] M. Stylianou, N. Murphy, L. R. Peraza, S. Graziadio, R. Cromarty, A. Killen, J. T. O'Brien, A. J. Thomas, F. E. N. LeBeau, and J.-P. Taylor, “Quantitative electroencephalography as a marker of cognitive fluctuations in dementia with Lewy bodies and an aid to differential diagnosis,” CLINICAL NEUROPHYSIOLOGY, vol. 129, pp. 1209-1220, June 2018.

[36] S. Gudmundsson, T. P. Runarsson, S. Sigurdsson, G. Eiriksdottir, and K. Johnsen, “Reliability of quantitative eeg features,” Clinical Neurophysiology, vol. 118, no. 10, pp. 2162-2171, 2007.

[37] J. Snaedal, G. H. Johannesson, T. E. Gudmundsson, S. Gudmundsson, T. H. Pajdak, and K. Johnsen, “The use of EEG in Alzheimer's disease, with and without scopolamine - A pilot study,” CLINICAL NEUROPHYSIOLOGY, vol. 121, pp. 836-841, June 2010.

[38] M. Johannsson, J. Snaedal, G. H. Johannesson, T. E. Gudmundsson, and K. Johnsen, “The Acetylcholine Index: An Electroencephalographic Marker of Cholinergic Activity in the Living Human Brain Applied to Alzheimer's Disease and Other Dementias,” DEMENTIA AND GERIATRIC COGNITIVE DISORDERS, vol. 39, no. 3-4, pp. 132-142, 2015.

[39] S. J. Colloby, R. A. Cromarty, L. R. Peraza, K. Johnsen, G. Jo'hannesson, L. Bonanni, M. Onofrj, R. Barber, J. T. O'Brien, and J.-P. Taylor, “Multimodal eeg-mri in the differential diagnosis of alzheimer's disease and dementia with lewy bodies,” Journal of psychiatric research, vol. 78, pp. 48-55, 2016.

[40] H. Helgadottir, 0. 0. Gudmundsson, G. Baldursson, P. Magnusson, N. Blin, B.

Brynjolfsdottir, A. Emilsdottir, G. B. Gudmundsdottir, M. Lorange, P. K. Newman, G. H. Johannesson, and K. Johnsen, “Electroencephalography as a clinical tool for diagnosing and monitoring attention deficit hyperactivity disorder: a cross-sectional study,” BMJ OPEN, vol. 5, no. 1, 2015.

[41] J. N. Caviness, C. H. Adler, J. G. Hentz, H. A. Shill, V. G. Evidente, E. D. Driver-Dunckley, M. N. Sabbagh, L. Sue, and T. G. Beach, “Incidental lewy body disease: electrophysiologi-cal findings suggesting pre-clinical lewy body disorders,” Clinical Neurophysiology, vol. 122, no. 12, pp. 2426-2432, 2011.

[42] C. H. Adler, D. J. Connor, J. G. Hentz, M. N. Sabbagh, J. N. Caviness, H. A. Shill, B. Noble, and T. G. Beach, “Incidental lewy body disease: clinical comparison to a control cohort,” Movement Disorders, vol. 25, no. 5, pp. 642-646, 2010.

[43] A. DelleDonne, K. J. Klos, H. Fujishiro, Z. Ahmed, J. E. Parisi, K. A. Josephs, R. Frigerio, M. Burnett, Z. K. Wszolek, R. J. Uitti, et al., “Incidental lewy body disease and preclinical parkinson disease,” Archives of neurology, vol. 65, no. 8, pp. 1074-1080, 2008.

[44] M. Nuwer, “Assessment of digital eeg, quantitative eeg, and eeg brain mapping: report of the american academy of neurology and the american clinical neurophysiology society,” Neurology, vol. 49, no. 1, pp. 277-292, 1997.

[45] G. S. Gronseth, L. M. Woodroffe, and T. S. Getchius, “Clinical practice guideline process man-ual,” 2011.

[46] G. McKhann, D. Drachman, M. Folstein, R. Katzman, D. Price, and E. M. Stadlan, “Clinical diagnosis of alzheimer's disease report of the nincds-adrda work group* under the auspices of department of health and human services task force on alzheimer's disease,” Neurology, vol. 34, no. 7, pp. 939-939, 1984.

[47] R Core Team, R: A Language and Environment for Statistical Computing. R Foundation for Statistical Computing, Vienna, Austria, 2017.

[48] H. Wickham, ggplot2: Elegant Graphics for Data Analysis. Springer-Verlag New York, 2009.

[49] H. Bengtsson, R.matlab: Read and Write MAT Files and Call MATLAB from Within R, 2016. R package version 3.6.1.

### EXAMPLE 2

### Sex- and Age-Based Differences in Brain Wave Patterns Measured with Electroencephalography (EEG)

Age is a known risk factor for dementia and brain changes due to age have been studied for decades¹⁻³. Over the last 5-10 years there has been an increased interest in sex differences in neuroscience in general⁴. Sex is defined as biological differences such as chromosomal, gonadal or hormonal differences. Age and sex differences have been reported in the brain at the structural, functional, and behavioral level in healthy individuals and both factors play an important role in the development and progression of diseases such as Alzheimer's disease^{1,4,5}.

Several studies have shown age-dependent changes in EEG features such as EEG rhythmic activity (e.g., gamma, beta, alpha, theta and delta) as well as changes in coherences in resting state EEG in the older population⁶⁻¹⁴. A recent study, including over 6000 participants investigated the age-related changes in EEG power throughout adulthood using a four channel EEG device. An overall age-related shift in band power was found, shifting from lower to higher frequencies, as well as a gradual slowing of peak a frequency with age¹⁵. Another study investigating the source of cortical rhythm reported that occipital delta and posterior cortical alpha rhythms decrease in magnitude during physiological aging with both linear and nonlinear trends¹⁶.

Numerous EEG studies have revealed sex differences in brain function in the general population as well as different patient populations¹⁵′¹⁷. In the general population females have been found to have a higher overall EEG power in most frequency bands^{15,19}, higher power in δ and α bands as well as in slow waves in females compared to men during sleep^{20,21}, higher overall β activity²², and δ, θ, α, and β bands during rest and photic stimulation^{23,24}. In patient populations EEG studies have shown sex differences in individual with attention deficit hyperactivity disorder (ADHD)²⁵ as well as other psychiatric disorders¹⁷.

Including Sex as a Feature

In order to demonstrate the effect on classifier performance due to taking sex into account as a feature, the impact on a prognostic classifier designed to separate mild cognitive impairment (MCI) subjects into subjects who convert into dementia (cMCI) versus subjects who remain cognitively stable (sMCI) was evaluated. The development of the classifiers relied on data gathered in a clinical trial where subjects 201 (89 males/112 females) visiting a memory clinic for the first time receiving a characterization of MCI. During the initial visit a baseline resting state EEG recording was performed. The subjects were then followed clinically for at least 3 years and up to 10 years, to determine which subjects were sMCI at baseline and which subjects were cMCI. The clinical findings are divided according to the table below.

| Group | Males | Females | Total |
|---|---|---|---|
| MCI | 89 | 112 | 201 |
| sMCI | 43 | 54 | 97 |
| cMCI | 46 | 58 | 104 |

The index considered were classifiers contrasting the sMCI group vs cMCI group for 3 different scenarios: male group only, female group only, and the combined male and female groups. The classifiers were developed as described above resulting in several genetic generations of classifier candidates for each group. Comparing the statistics of the classifier candidate's performance in terms of the estimated AUC reveals the performance benefit of considering the sexes independently (FIG. 10).

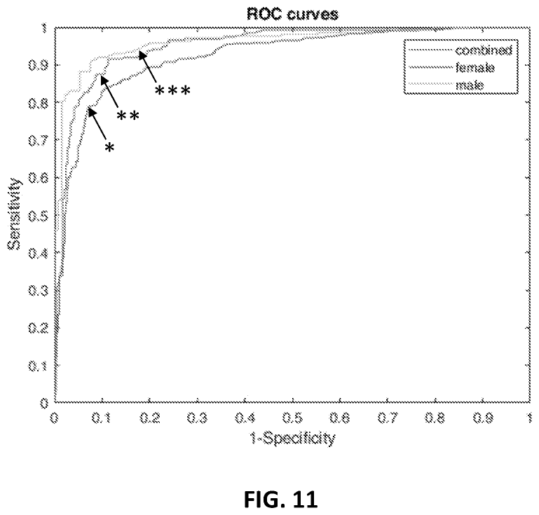

To demonstrate the benefit of considering the sex of the subjects whose EEG is to be analyzed, three cross-validation experiments were performed and the resulting ROC statistics were considered. EEG recordings from 2 cohorts were analyzed—a group of healthy volunteers and a group of subjects diagnosed with dementia. Results are shown in FIG. 11. First, sex was disregarded, resulting in the curve indicated by a single asterisk. Then females and males were considered separately, resulting in the curves indicated by double and triple asterisks, respectively. The results show that it is beneficial to treat the sexes separately as both sensitivity and specificity is significantly higher at the optimal cut-off point on the curves, which is found closest to the upper left-hand corner in the graph.

Including Biological Age as a Feature

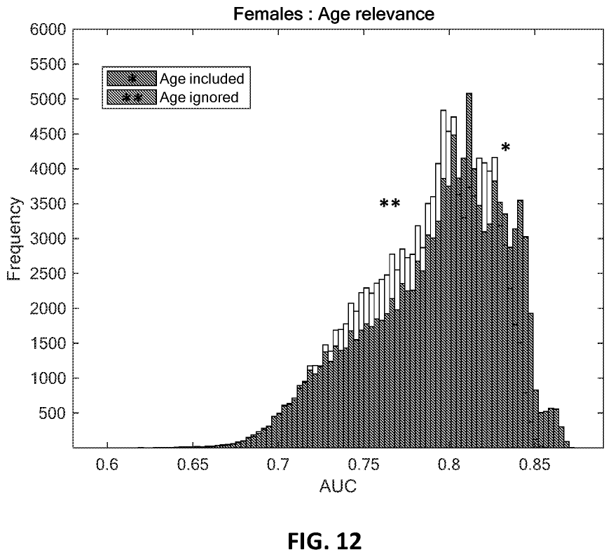

Continuing with the example above we considered the female group specifically. To demonstrate the performance benefit of including biological age as a feature in the classifiers we compare statistically the genetic evolution generation for the case when age is not considered and the case where age is included. The former case is the same as demonstrated for the sex relevance example. Age was not forced into the classifier candidates in the second case but included as a possible feature contributing to the classifier. We found that the AUC statistics for the genetic evolution generated classifiers revealed that there is a significant benefit to the classifier performance to include the age as a feature as is illustrated in FIG. 12.

### References

1. Mielke, M. M., Vemuri, P. & Rocca, W. A. Clinical epidemiology of Alzheimer's disease: assessing sex and gender differences. Clin. Epidemiol. 6,37-48 (2014).

2. Lindsley, D. B. A Longitudinal Study of the Occipital Alpha Rhythm in Normal Children: Frequency and Amplitude Standards. Pedagog. Semin. J. Genet. Psychol. 55, 197-213 (1939).

3. Hedden, T. & Gabrieli, J. D. E. Insights into the ageing mind: a view from cognitive neuroscience. Nat. Rev. Neurosci. 5, 87-96 (2004).

4. Cahill, L. Why sex matters for neuroscience. Nat. Rev. Neurosci. 7, 477-484 (2006).

5. Grady, C. The cognitive neuroscience of ageing. Nat. Rev. Neurosci. 13, 491-505 (2012).

6. Matthis, P., Scheffner, D., Benninger, Chr., Lipinski, Chr. & Stolzis, L. Changes in the background activity of the electroencephalogram according to age. Electroencephalogr. Clin. Neurophysiol. 49, 626-635 (1980).

7. Clarke, A. R., Barry, R. J., McCarthy, R. & Selikowitz, M. Age and sex effects in the EEG: development of the normal child. Clin. Neurophysiol. Off. J. Int. Fed. Clin. Neurophysiol. 112, 806-814 (2001).

8. Marshall, P. J., Bar-Haim, Y. & Fox, N. A. Development of the EEG from 5 months to 4 years of age. Clin. Neurophysiol. Off. J. Int. Fed. Clin. Neurophysiol. 113, 1199-1208 (2002).

9. Cragg, L. et al. Maturation of EEG power spectra in early adolescence: a longitudinal study. Dev. Sci. 14, 935-943 (2011).

10. Al Zoubi, O. et al. Predicting Age From Brain EEG Signals-A Machine Learning Approach. Front. Aging Neurosci. 10, (2018).

11. Gasser, T., Verleger, R., Bächer, P. & Sroka, L. Development of the EEG of school-age children and adolescents. I. Analysis of band power. Electroencephalogr. Clin. Neurophysiol. 69, 91-99 (1988).

12. Kikuchi, M., Wada, Y., Koshino, Y., Nanbu, Y. & Hashimoto, T. Effect of normal aging upon interhemispheric EEG coherence: analysis during rest and photic stimulation. Clin. EEG Electroencephalogr. 31, 170-174 (2000).

13. Marciani, M. G. et al. Quantitative EEG evaluation in normal elderly subjects during mental processes: age-related changes. Int. J. Neurosci. 76, 131-140 (1994).

14. Widagdo, M. M., Pierson, J. M. & Helme, R. D. Age-related changes in qEEG during cognitive tasks. Int. J. Neurosci. 95, 63-75 (1998).

15. Hashemi, A. et al. Characterizing Population EEG Dynamics throughout Adulthood. eNeuro 3, (2016).

16. Babiloni, C. et al. Sources of cortical rhythms in adults during physiological aging: a multicentric EEG study. Hum. Brain Mapp. 27, 162-172 (2006).

17. Calzada-Reyes, A., Alvarez-Amador, A., Galán-García, L. & Valdés-Sosa, M. Sex differences in QEEG in adolescents with conduct disorder and psychopathic traits. Ann. Clin. Neurophysiol. 21, 16 (2019). 18. Veldhuizen, R. J., Jonkman, E. J. & Poortvliet, D. C. J. Sex differences in age regression parameters of healthy adults-normative data and practical implications. Electroencephalogr. Clin. Neurophysiol. 86, 377-384 (1993).

19. Horita, M., Takizawa, Y., Wada, Y., Futamata, H. & Hashimoto, T. [Sex differences in EEG background activity: a study with quantitative analysis in normal adults]. Rinsho Byori 43, 177-180 (1995).

20. Latta, F., Leproult, R., Tasali, E., Hofmann, E. & Van Cauter, E. Sex differences in delta and alpha EEG activities in healthy older adults. Sleep 28, 1525-1534 (2005).

21. Mourtazaev, M. S., Kemp, B., Zwinderman, A. H. & Kamphuisen, H. A. Age and gender affect different characteristics of slow waves in the sleep EEG. Sleep 18, 557-564 (1995).

22. Mundy-Castle, A. C. Theta and beta rhythm in the electroencephalograms of normal adults. Electroencephalogr. Clin. Neurophysiol. 3, 477-486 (1951).

23. Wada, Y., Takizawa, Y., Jiang, Z. Y. & Yamaguchi, N. Gender differences in quantitative EEG at rest and during photic stimulation in normal young adults. Clin. EEG Electroencephalogr. 25, 81-85 (1994).

24. Carrier, J., Land, S., Buysse, D. J., Kupfer, D. J. & Monk, T. H. The effects of age and gender on sleep EEG power spectral density in the middle years of life (ages 20-60 years old). Psychophysiology 38, 232-242 (2001).

25. Hermens, D. F., Kohn, M. R., Clarke, S. D., Gordon, E. & Williams, L. M. Sex differences in adolescent ADHD: findings from concurrent EEG and EDA. Clin. Neurophysiol. 116, 1455-1463 (2005).

### EXAMPLE 3

### Harmonization of Features Based on the EEG Recording Equipment

The frequency response of different types of EEG recorders varies. FIG. 13 illustrates how equipment from different manufacturers and type respond to signals at different frequencies within the relevant frequency range for EEG recordings. The characteristic response curves are measured by feeding a sinusoidal signal of known amplitude and frequency into the equipment with a signal generator. This is done by fixing amplitude of the signal and then stepping through the relevant frequency range at, say steps of 0.5Hz. The amplitude measured by the equipment is then compared to the reference signal and the power response is deduced by the squared ratio of the measured signal to the reference signal at that frequency. For a specific piece of equipment, this procedure results in power response curve pᵢ where i is the frequency. Examples of response curves are shown in FIG. 13. In this example, harmonization of features estimated by the equipment is achieved by scaling the resulting Fourier components stored in the SPC format, σ_{cij}. The scaling is achieved by σ_{cij}^{sc}=σ_{cij}/\sqrt{pᵢ}. Further estimates based on the FFT components are then done using the scaled components.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A computer-implemented method for producing an index, comprising:
conditioning, using one or more processors, electroencephalographic (EEG) signals present in an EEG recording previously obtained from an individual having dementia;
determining, using the one or more processors, frequency domain features from the conditioned EEG signals;
determining, using the one or more processors, connectivity features from the frequency domain features, wherein the connectivity features comprise connectivity features determined from a frequency range of from 35 Hz to 45 Hz divided into two or more sub-bands; and
producing, using the one or more processors, an index calculated at least in part as a function of one or more of the connectivity features determined from a frequency range of from 35 Hz to 45 Hz divided into two or more sub-bands with varying contribution to the calculation of the index.

2. The method according to claim 1, wherein the index is calculated as a function of from 5 to 20 connectivity features.

3. The method according to claim 1 or claim 2, wherein the index is based on a linear combination of the connectivity features.

4. The method according to any one of claims 1 to 3, wherein the sub-bands are defined with a frequency resolution of from 0.2 Hz to 5 Hz.

5. The method according to any one of claims 1 to 4, wherein, in addition to one or more of the connectivity features, the index is calculated as a function of one or more of the frequency domain features.

6. The method according to any one of claims 1 to 5, wherein the index is further calculated using the sex of the individual as a feature, the age of the individual as a feature, or both.

7. The method according to any one of claims 1 to 6, comprising harmonizing the frequency domain features based on the type of EEG recording equipment used to obtain the EEG recording.

8. A computer-implemented method for producing an index, comprising:
conditioning, using one or more processors, electroencephalographic (EEG) signals present in an EEG recording previously obtained from an individual having dementia;
determining, using the one or more processors, frequency domain features from the conditioned EEG signals;
determining, using the one or more processors, connectivity features from the frequency domain features; and
producing, using the one or more processors, an index calculated at least in part as a function of one or more of the connectivity features, and wherein the index is further calculated as a function of the age of the individual, the sex of the individual, or both.

9. A computer-implemented method for producing an index, comprising:
conditioning, using one or more processors, electroencephalographic (EEG) signals present in an EEG recording previously obtained from an individual having dementia;
determining, using the one or more processors, frequency domain features from the conditioned EEG signals;
determining, using the one or more processors, connectivity features from the frequency domain features;
harmonizing, using the one or more processors, the frequency domain features based on the type of EEG recording equipment used to obtain the EEG recording; and
producing, using the one or more processors, an index calculated at least in part as a function of one or more of the connectivity features and the harmonized frequency domain features.

10. The method according to any one of claims 1 to 9, further comprising generating a report comprising the index.

11. The method according to claim 10, wherein generating a report comprises displaying the index on a display or printout.

12. The method according to claim 11, wherein the index is displayed graphically in context with data from:
a database of individuals having dementia and a database of individuals not having dementia;
a database of individuals having a particular type of dementia and a database of individuals not having dementia; and/or
a database of individuals having a first type of dementia and a database of individuals having a second type of dementia.

13. The method according to claim 12, wherein the index is displayed graphically in context with data from a database of individuals having a first type of dementia and a database of individuals having a second type of dementia, and wherein the first and second types of dementia are selected from the group consisting of: a Lewy Body Dementia, Dementia with Lewy Bodies, Parkinson's Disease Dementia, Alzheimer's Disease Dementia, Frontal Lobe Dementia, and Vascular Dementia.

14. The method according to claim 13, wherein the first type of dementia is a Lewy Body Dementia and the second type of dementia is Alzheimer's Disease Dementia.

15. The method according to any one of claims 1 to 14, further comprising diagnosing the individual as having dementia based at least in part on the index.

16. The method according to claim 15, further comprising treating the individual's dementia based on the diagnosis.

17. A computer-implemented method for producing an index, comprising:
conditioning, using one or more processors, electroencephalographic (EEG) signals present in an EEG recording previously obtained from an individual;
determining, using the one or more processors, frequency domain features from the conditioned EEG signals;
determining, using the one or more processors, connectivity features from the frequency domain features, wherein the connectivity features comprise connectivity features determined from a frequency range of from 35 Hz to 45 Hz divided into two or more sub-bands;
producing, using the one or more processors, an index calculated at least in part as a function of one or more of the connectivity features determined from a frequency range of from 35 Hz to 45 Hz divided into two or more sub-bands with varying contribution to the calculation of the index; and
predicting the onset of dementia in the individual based at least in part on the index.

18. The method according to any one of claims 1 to 17, further comprising, prior to the conditioning, collecting EEG signals from the individual to obtain the EEG recording.

19. A non-transitory computer readable medium comprising instructions, when executed by one or more processors, cause the one or more processors to perform the method according to any one of claims 1 to 18.

20. A computer device, comprising:
one or more processors; and
the non-transitory computer readable medium of claim 19.
